(19) United States
(12) Patent Application Publication
(10) Pub. No.: US 2018/0153872 A1
(43) Pub. Date: Jun. 7, 2018

(54) HETEROARYL CARBONITRILES FOR THE TREATMENT OF DISEASE

(71) Applicants: Phillip FROST, Miami Beach, FL (US); OPKO Health, Inc., Miami, FL (US)

(72) Inventors: Jane H. HSIAO, Miami, FL (US); Phillip FROST, Miami Beach, FL (US)

(21) Appl. No.: 15/736,950

(22) PCT Filed: Jul. 12, 2016
(86) PCT No.: PCT/US16/41876

§ 371 Date: Dec. 15, 2017

Related U.S. Application Data
(60) Provisional application No. 62/192,772, filed on Jul. 15, 2015.

(51) Int. Cl. A61K 31/454; A61P 17/04

(52) U.S. Cl. CPC A61K 31/454; A61P 17/04

(57) ABSTRACT

The invention relates to pharmaceutical compositions and dosage forms having a piperidine carbonitrile as an active ingredient for the treatment of various diseases and conditions. The preferred compound is a crystalline form of a trifluoromethylphenylalkoxyalkylheteroarylaryl piperidine carbonitrile. Methods of treating diseases and conditions are disclosed as well as processes to make crystalline forms of the above compounds.

This present application is a 371 National filing of PCT Application No. PCT/US2016/041876 filed Jul. 12, 2016, which claims priority of U.S. provisional patent application No. 62/192,772 filed Jul. 15, 2015, which are all incorporated herein by reference in their entirety.

### FIELD OF THE INVENTION

### Background of the Invention

Pruritus or severe itching of the skin is associated with a number of diseases and conditions including dry skin, pregnancy and skin diseases. The condition can be associated with skin conditions such as eczema (dermatitis), psoriasis, scabies, lice, chicken pox and hives. In addition, liver disease, celiac disease, kidney failure, iron deficiency anemia and thyroid conditions as well as certain cancers can include severe itching. Other diseases, conditions or causes of severe itching include nerve disorders, allergic reactions and side effects to certain drugs. It has been estimated that up to 23% of all adults suffer or may suffer from chronic pruritus and this condition is a burden on patients worldwide. Matterne et al., (2011) Prevalence, correlates and characteristics of chronic pruritus: a population based cross-sectional study, Acta Derm Venereol 91:674-679. Typical treatments for this condition include antihistamines, topical steroids and antibiotics. The treatment for chronic pruritus may also include opioid antagonists and antidepressants. None of these regimens result in complete resolution of the symptoms. There is a need for new drugs to treat pruritus and prurigo nodularis. While the scientific literature has suggested some association with NK1, substance P and many physiological and pathophysiological processes including pain, vomiting reflex, depression, anxiety, cardiovascular tone, salivary secretion, vasodilation, cell proliferation, immune and inflammatory responses and a host of other functions in various organs including the skin, the causes and/or modes of treatment of acute and chronic itching as well as other diseases such as alcohol dependency and depression are complex. In addition, the function or effects of substance P/NKR1 in the skin cross or encompass a range of cells and/or structures including epidermal dendritic cells, fibroblasts, hair follicles, keratinocytes, mast cells and melanoma—these effects include production of IFN-gamma, IL-1beta and IL-8; stress induced hair growth; production of NGF, leukotriene B4, IFN-gamma, IL-1beta and IL-8; release of histamine, leukotriene B4, prostaglandin D2 or tumor necrosis factor alpha, increased expression of NK1R and suppression of melanogenesis. SP is known to play a role in the skin of inducing neurogenic inflammation. MC degranulation results in the release of histamine, leukotriene B4, etc. and this can result in the induction of pruritus. Ikoma, et al. (2006) The Neurobiology of Itch. Nat Rev Neurosci 7: 535-547. See also, Stander, et al., NK-1 Antagonists and Itch, A. Cowan, G. Yosipovich (eds.), Pharmacology of Itch, Handbook of Experimental Pharmacology: 237-255, Springer Verlag (2015).

NK-1 antagonists are in development and on the market for the treatment of emesis and nausea associated with the use of chemotherapeutic drugs. These drugs include, for example, EMEND® (aprepitant) and rolapitant. Other NK-1 antagonists have been in development for the treatment of, for example, overactive bladder. Once such drug, serlopitant, an NK-1, substance P receptor antagonist previously known as MK-0594, was dropped from development for the treatment of overactive bladder and subsequently licensed and has undergone clinical trials for the treatment of pruritus. U.S. Pat. No. 8,906,951 discloses the use of VPD-737 (formerly MK-0594) for such treatment. This compound is disclosed as having an NK-1 Kd of 46 μM and it displaces substance P at the same receptor with an IC₅₀ of 61 μM.

Another NK-1 antagonist, aprepitant, was studied in patients having pruritic drug reactions to antineoplastic drugs. In several patients, application or provision of aprepitant resulted in a decrease in pruritus on the visual analogue scale (VAS). See Vincenzi et al. (2010a). Aprepitant against pruritus in patients with solid tumours. Support Care Cancer 18: 1229-1230 and Vincenzi et al. (2010b) Aprepitant for erlotinib-induced pruritus. N Engl J. Med 363:397-398. Mir et al. Aprepitant for pruritus: drug-drug interactions matter. Lancet Oncol 13: 964-965. Santani et al. (2012) Aprepitant for management of sever pruritus related to biological cancer treatments: a pilot study. Lancet Oncol 13: 1020-1024. Aprepitant was also studied in patients having chronic pruritus. It has been reported that the most significant response rate for treatment with aprepritant was in patients having atopic predisposition prurigo nodularis (PN). See Stander et al (2012) Medical treatment of pruritus. Expert Opin Emerg Drugs 17:335-345.

Compounds generally described as having formula I as described and disclosed in U.S. Pat. Nos. 7,709,641 and 8,026,341 are known NK-1 receptor antagonists. Such compounds are described as being useful in various disorders including emesis, depression, anxiety, inflammation and cough. The present invention relates to the use of these compounds in the treatment of pruritus and prurigo nodularis. In particular, the present invention relates to the use of (trifluoromethyl)phenyl]ethoxy}methyl)-3-(heterocyclic)-arylpiperidine compounds in the treatment of pruritus and prurigo nodularis. The present invention also relates to a crystalline form of a compound of formula I or II and its use in the treatment of NK-1 related diseases and conditions including cough, overactive bladder, alcohol dependency and depression.

### SUMMARY OF THE INVENTION

The present invention relates to the use of a compound of formula I or a pharmaceutically acceptable salt or solvate thereof for the treatment of pruritus and prurigo nodularis. The compound of formula I has the formula:

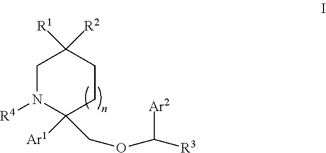

wherein:
R¹ and R² are independently selected from the group consisting of H, alkyl, haloalkyl, alkyl substituted with one or more hydroxyl groups, —CN, alkynyl, —N(R⁶)₂, —N(R⁶)—S(O₂)alkyl, —N(R⁶)—C(O)—N(R⁹)₂, -alkylene-CN, cycloalkylene-CN, -alkylene-O-alkyl, —C(O)-alkyl, —C(═N—OR⁵)-alkyl, —C(O)—N(R⁹)₂, —C(O)—O-alkyl, -alkylene-C(O)-alkyl, -alkylene-C(O)—O-alkyl, -alkylene-C(O)—N(R⁹)₂,
With the proviso that at least one of R¹ and R² is —CN,

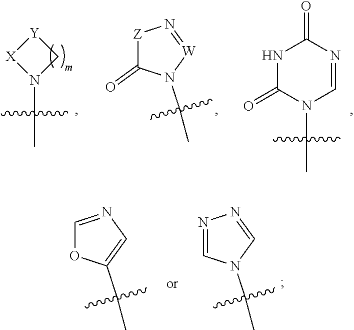

### W is ═C(R⁸)— or ═N—;

### X is —C(O)— or —S(O₂)—;

Y is selected from the group consisting of —CH₂—, —O—, and —N(R⁶)—C(O)—, with the proviso that:
(a) The nitrogen atom of —N(R⁶)—C(O)— is bonded to X, and
(b) If R¹ and/or R² is

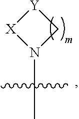

### And Y is —O—, X is not —S(O₂)—;

Z is —C(R⁷)₂—, —N(R⁶)—, or —O—;
R³ is selected from the group consisting of H, —CH₂OR⁵, and alkyl;
R⁴ is selected from the group consisting of H, alkyl, cycloalkyl, heterocycloalkyl, heteroaryl, aryl, acyl, aroyl, alkylsulfonyl, and arylsulfonyl;
R⁵ is H or alkyl;
R⁶ is selected from the group consisting of H, alkyl, cycloalkyl, and aryl;
Each R⁷ is independently H or alkyl; or
Each R⁷, together with the ring carbon to which they are shown attached, form a cycloalkylene ring;
R⁸ is selected from the group consisting of H, alkyl, alkyl substituted with one or more hydroxyl groups, —N(R⁶)₂, —N(R⁶)—S(O₂)-alkyl, —N(R⁶)—S(O₂)-aryl, —N(R⁶)—C(O)-alkyl, —N(R⁶)—C(O)-aryl, alkylene-O-alkyl, and —CN;
R⁹ is selected from the group consisting of H, alkyl, and aryl, or each R⁹, together with the nitrogen to which they are shown attached, form a heterocycloalkyl ring;
Ar¹ and Ar² are each independently selected from the group consisting of unsubstituted aryl and aryl substituted with 0 to 3 substituents selected from the group consisting of halogen, alkyl, alkoxy, haloalkyl, haloalkoxy, —CN, —OH, and —NO₂;
n is 0, 1, or 2; and
m is 1, 2 or 3.

The present invention comprises:

A pharmaceutical composition comprising:
a) a compound of Formula II

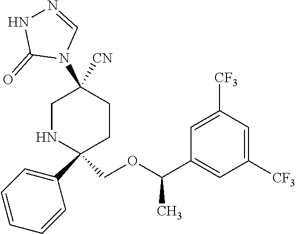 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable salt thereof and
b) a pharmaceutically acceptable vehicle selected from the group consisting of a diluent, binder, disintegrant, wetting agent or lubricant.

The invention also comprises a composition according to the above formulation in an oral dosage form;

a composition according to the above formulation having 2.5 mg to 250 mgs of a compound of formula II in the free amine form;
a composition according to the above formulation in the form of a capsule, tablet or suspension.

The invention comprises a method for treating chronic cough in a patient in need of treatment, the method comprising administering to the patient an effective amount of the pharmaceutical composition described herein.

The invention also comprises, a method for treating overactive bladder (OAB) in a patient in need of treatment thereof, the method comprising administering to the patient an effective amount of the pharmaceutical composition having a compound of formula II.

The invention further comprises a method for treating depression in a patient in need of treatment thereof, the method comprising administering to the patient an effective amount of the pharmaceutical composition having a compound of formula II and a method for treating alcohol dependency in a patient in need of treatment thereof, the method comprising administering to the patient an effective amount of a composition comprising a compound of formula II.

In a preferred embodiment, the invention comprises a method for treating pruritis in a patient in need of treatment thereof, the method comprising administering to the patient an effective amount of a pharmaceutical composition comprising a compound of formula II and a method for treating prurigo nodularis in a patient in need of treatment thereof, the method comprising administering to the patient an effective amount of a pharmaceutical composition comprising a compound of formula II.

The invention further comprises a combination comprising a pharmaceutically effective amount of a formulation according to claim 1 and at least one additional active pharmaceutical ingredient selected from the group consisting of an antidepressant, an anti-nausea or anti-emetic medication, a chemotherapeutic agent or an anti-inflammatory agent.

The invention also comprises a combination wherein the antidepressant is selected from an SSRI.

The invention also comprises a combination wherein the anti-nausea/anti-emetic medication is selected from a 5-HT3 receptor antagonist.

The invention further comprises a crystalline form of a compound having the formula:

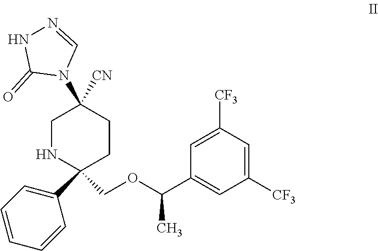 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable salt thereof.

The invention comprises a crystalline form of formula II as the free amine and which is non-hygroscopic.

The invention comprises a crystalline form of formula II in the form of a powder.

The invention comprises a pharmaceutical composition comprising a compound of formula II in crystalline form and a pharmaceutically acceptable excipient.

The invention comprises a pharmaceutical composition comprising a crystalline compound of formula II and a pharmaceutically acceptable excipient wherein the excipients are selected from the group consisting of a diluent, binder, disintegrant, wetting agent or lubricant.

The invention comprises an amorphous form of a compound of formula II or a pharmaceutically acceptable salt thereof.

The invention comprises an amorphous salt form of a compound of formula II wherein the salt is a hydrochloride salt or a tosylate salt.

The invention comprises a method of treating acute and delayed nausea and vomiting associated with initial and repeat courses of highly emetogenic cancer chemotherapy comprising administration of an intravenous formulation of a compound of formula II in combination with at least one additional antiemetic agent.

The invention comprises a method of treating acute and delayed nausea and vomiting associated with initial and repeat courses of moderately emetogenic cancer chemotherapy comprising administration of an intravenous formulation of a compound of formula II in combination with at least one additional antiemetic agent.

The invention comprises a method of treating emesis in a patient in need of treatment thereof comprising administration of a pharmaceutically effective amount of a crystalline compound of formula II.

The invention comprises administering a bolus dose of a compound of formula II in a pharmaceutical composition as recited herein.

The invention comprises a compound of Formula IIa, IIb and IIc and pharmaceutically acceptable salts thereof:

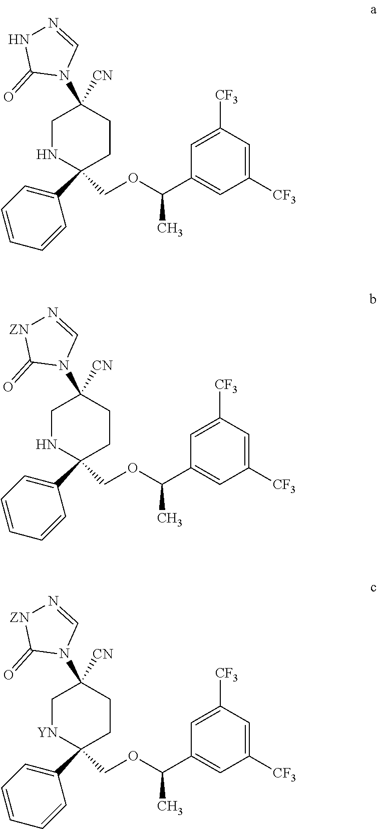

wherein Z and Y are independently selected from the group consisting of —PO(OH)O-M+, —PO(O⁻)22M+, —PO(O⁻)₂D²+, —[C(R¹)(R²)]n-PO(OH)O-M+, —[C(R¹)(R²)]n-PO(O⁻)22M+, —[C(R¹)(R²)]n-PO(O⁻)₂D²+, —C(O)[C(R¹)(R²)]m-OPO(O⁻)22M+, —C(O)[C(R¹)(R²)]oNR¹R², —C(O)[C(R¹)(R²)]pCO₂-M+, —SO₃-M+, —[C(R¹)(R²)]qSO₃-M+ and —[C(R¹)(R²)]rOC(O)OR³, wherein R³ is selected from the group consisting of

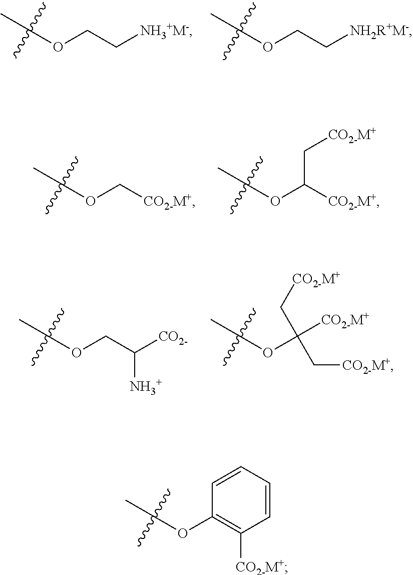

M⁺ is selected from a monovalent cation; D⁺ is selected from a divalent cation; R¹ and R² are independently selected from H or C₁₋₆ alkyl; n is 1-4; m, o, and p are independently selected from 0-4; and R is selected from C₁₋₆ alkyl.

The invention comprises a compound of formula IIa, IIb or IIc wherein Z is selected from H and Y is selected from any one of the groups shown above for Z and Y exclusive of H.

The invention comprises a compound of formula IIa, IIb or IIc wherein M+ is selected from ammonium salts, alkali metal salts such as sodium, alkaline earth metal salts such as calcium and magnesium, salts with organic bases such as N-methyl-D-glucamine or dicyclohexylamine or amino acid salts such as arginine or lysine.

The invention also comprises a method of treating acute and delayed nausea and vomiting associated with initial and repeat courses of highly emetogenic cancer chemotherapy comprising administration of an intravenous formulation of a compound of formula IIa, IIb or IIc in combination with at least one additional antiemetic agent.

The present invention comprises a method of treating acute and delayed nausea and vomiting associated with initial and repeat courses of moderately emetogenic cancer chemotherapy comprising administration of an intravenous formulation of a compound of formula IIa, IIb or IIc in combination with at least one additional antiemetic agent.

The invention comprises an intravenous formulation comprising a compound of formula IIa, IIb or IIc.

The invention comprises a method of treating a patient in need of treatment thereof for CINV comprising administering an intravenous formulation having a compound of formula II wherein the formulation is administered at one dose per chemotherapeutic treatment cycle.

The invention comprises a pharmaceutical intravenous formulation comprising a compound of formula II or a pharmaceutically acceptable salt thereof wherein the T½ for the active ingredient is about 10-13 days and wherein upon administration the blood concentration (ng/mL) for doses ranging from about 50 to about 200 mg and directly after administration ranges from about 280 ng/mL to about 850 ng/mL.

The invention comprises a micelle formulation suitable for intravenous administration comprising a compound of formula II or pharmaceutically acceptable salts thereof.

The invention comprises an emulsion formulation suitable for intravenous administration comprising a compound of formula II or a pharmaceutically acceptable salt thereof.

The invention comprises a pharmaceutical composition comprising a compound of formula II or pharmaceutically acceptable salts thereof and a non-ionic solubilizer.

The invention comprises a pharmaceutical formulation comprising a compound of formula II and having stability for up to eighteen months when stored at a temperature range of 15-30 degrees C.

The invention comprises an aqueous suspension comprising a compound of formula II and a cellulosic polymer.

The invention comprises a suspension comprising a compound of formula II and a cellulosic polymer wherein the polymer is selected from the group consisting of hydroxypropylmethylcellulose.

### DETAILED DESCRIPTION OF THE INVENTION

The present invention relates to the use of a compound of formula I or a pharmaceutically acceptable salt or solvate thereof for the treatment of pruritus. The compound of formula I has the formula:

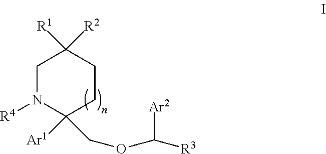

wherein:
R¹ and R² are independently selected from the group consisting of H, alkyl, haloalkyl, alkyl substituted with one or more hydroxyl groups, —CN, alkynyl, —N(R⁶)₂, —N(R⁶)—S(O₂)alkyl, —N(R⁶)—C(O)—N(R⁹)₂, -alkylene-CN, cycloalkylene-CN, -alkylene-O-alkyl, —C(O)-alkyl, —C(═N—OR⁵)-alkyl, —C(O)—N(R⁹)₂, —C(O)—O-alkyl, -alkylene-C(O)-alkyl, -alkylene-C(O)—O-alkyl, -alkylene-C(O)—N(R⁹)₂,
With the proviso that at least one of R¹ and R² is —CN,

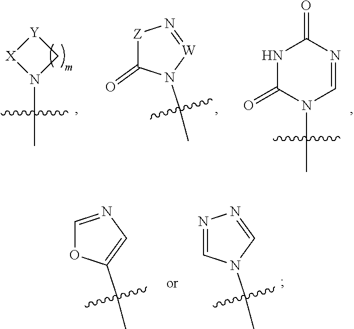

### W is ═C(R⁸)— or ═N—;

### X is —C(O)— or —S(O₂)—;

Y is selected from the group consisting of —CH₂—, —O—, and —N(R⁶)—C(O)—, with the proviso that:
c) The nitrogen atom of —N(R⁶)—C(O)— is bonded to X, and
d) If R¹ and/or R² is

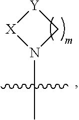

### And Y is —O—, X is not —S(O₂)—;

Z is —C(R⁷)₂—, —N(R⁶)—, or —O—;
R³ is selected from the group consisting of H, —CH₂OR⁵, and alkyl;
R⁴ is selected from the group consisting of H, alkyl, cycloalkyl, heterocycloalkyl, heteroaryl, aryl, acyl, aroyl, alkylsulfonyl, and arylsulfonyl;
R⁵ is H or alkyl;
R⁶ is selected from the group consisting of H, alkyl, cycloalkyl, and aryl;
Each R⁷ is independently H or alkyl; or
Each R⁷, together with the ring carbon to which they are shown attached, form a cycloalkylene ring;
R⁸ is selected from the group consisting of H, alkyl, alkyl substituted with one or more hydroxyl groups, —N(R⁶)₂, —N(R⁶)—S(O₂)-alkyl, —N(R⁶)—S(O₂)-aryl, —N(R⁶)—C(O)-alkyl, —N(R⁶)—C(O)-aryl, alkylene-O-alkyl, and —CN;
R⁹ is selected from the group consisting of H, alkyl, and aryl, or each R⁹, together with the nitrogen to which they are shown attached, form a heterocycloalkyl ring;
Ar¹ and Ar² are each independently selected from the group consisting of unsubstituted aryl and aryl substituted with 0 to 3 substituents selected from the group consisting of halogen, alkyl, alkoxy, haloalkyl, haloalkoxy, —CN, —OH, and —NO₂;
n is 0, 1, or 2; and
m is 1, 2 or 3.

In yet another embodiment, the compounds of the formula I, wherein the variables are as described above and

R³ is C₁₋₆ alkyl;

### R⁴ is H;

Ar¹ is phenyl;
Ar² is phenyl substituted with 1 to 3 substituents selected from the group consisting of halogen, C₁₋₆ alkyl, C₁₋₆ alkoxy, C₁₋₆ haloalkyl, C₁₋₆ haloalkoxy, —CN, and —NO₂; and n is 1 are preferred in a method of treating pruritus.

In another embodiment to treat pruritus, a compound of formula I have the following structure IA:

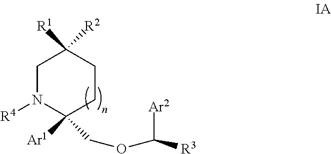

Wherein the variables are as described above, or, in a preferred embodiment,
R¹ and R² are independently selected from the group consisting of H, alkyl, haloalkyl, alkyl substituted with one or more hydroxyl groups, —CN, alkynyl, —N(R⁶)₂, —N(R⁶)—S(O₂)alkyl, —N(R⁶)—C(O)—N(R⁹)₂, -alkylene-CN, cycloalkylene-CN, -alkylene-O-alkyl, —C(O)-alkyl, —C(═N—OR⁵)-alkyl, —C(O)—N(R⁹)₂, —C(O)—O-alkyl, -alkylene-C(O)-alkyl, -alkylene-C(O)—O-alkyl, -alkylene-C(O)—N(R⁹)₂,
With the proviso that at least one of R¹ and R² is —CN,

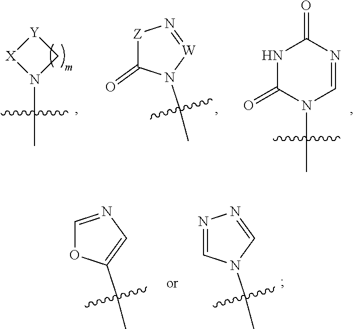

### W is ═C(R⁸)— or ═N—;

### X is —C(O)— or —S(O₂)—;

Y is selected from the group consisting of —CH₂—, —O—, and —N(R⁶)—C(O)—, with the proviso that:
a) The nitrogen atom of —N(R⁶)—C(O)— is bonded to X, and
b) If R¹ and/or R² is

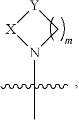

### And Y is —O—, X is not —S(O₂)—;

Z is —C(R⁷)₂—, —N(R⁶)—, or —O—;
R³ is C₁₋₆ alkyl;

### R⁴ is H;

Ar¹ is phenyl;
Ar² is phenyl substituted with 1 to 3 substituents selected from the group consisting of halogen, C₁₋₆ alkyl, C₁₋₆ alkoxy, C₁₋₆ haloalkyl, C₁₋₆ haloalkoxy, —CN, and —NO₂; and n is 1 and pharmaceutically acceptable salts and/or solvates thereof.

In another embodiment, the compounds of formula IA are used in the treatment of pruritus wherein

R¹ and R² are each independently selected from the group consisting of H, —CH₃, —CH₂—CH₂—CH₃, —CH₂C¹, —CH₂F, —CHC¹², —CHF², —CF³, —CH₂OH, —CH₂CH₂OH, —CH₂CH(OH)CH₃, —CH₂C(OH)(CH₃)₂, —CN, —CH₂CN, —NH₂, —NH—S(O₂)—CH₃, —NH—C(O)—NH₂, —CH₂OCH₃, —C(O)CH₃, —C(O)CH₂CH₃, —C(═N—OH)—CH₃, —C(═N—OH)—CH₂CH₃, —C(═N—OCH₃)—CH₃, —C(O)—NH₂, —C(O)NH(CH₃), —C(O)—O—CH₃, —CH₂—C(O)O—CH₃, —CH₂—C(O)OCH₂CH₃, —CH₂C(O)—NH(CH₂CH₃, —CH₂C(O)—NH₂,

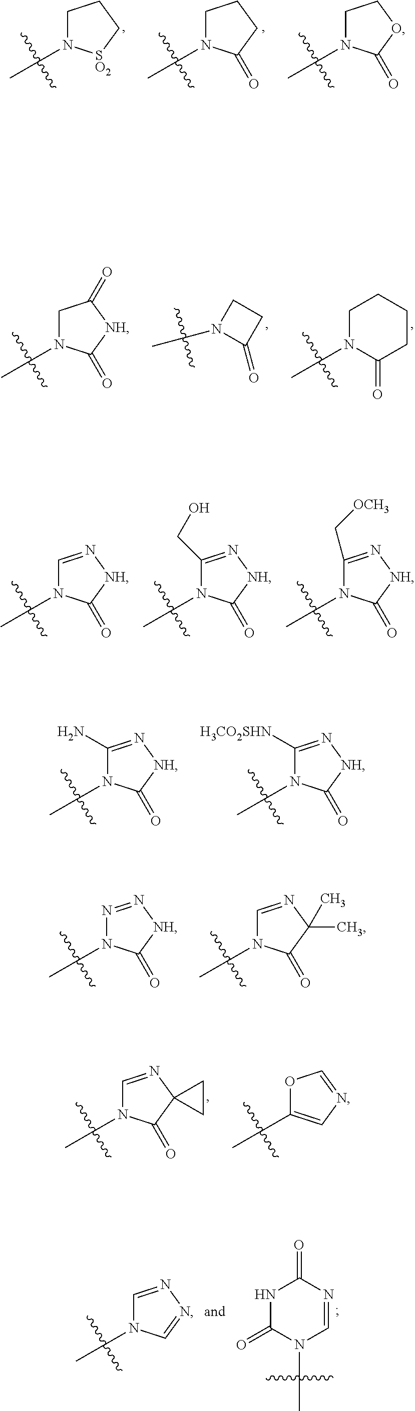

R³ is —CH₃;

### R⁴ is H;

Ar¹ is phenyl;
Ar² is phenyl substituted with 1 to 3 substituents selected from the group consisting of halogen, C₁₋₆ alkyl, C₁₋₆ haloalkyl, C₁₋₆ haloalkoxy, —CN, and —NO₂; and n is 1 and pharmaceutically acceptable salts and/or solvates thereof. In a preferred embodiment, Ar² is a phenyl group substituted with 1 to 3 substituents selected from CH₂F, CHF₂, or CF₃. U.S. Pat. Nos. 7,709,641 and 8,026,364 are incorporated by reference in their entirety. The term “alkyl” means an aliphatic hydrocarbon group which may be straight or branched and comprise 1 to 12 carbon atoms, with 1 to 6 being most preferred. The term “substituted alkyl” means that the alkyl group may be substituted by one or more substituents. The term “alkylene” means a divalent aliphatic hydrocarbon group which may be straight or branched and comprises 1 to 12 carbon atoms, with 1 to 6 being most preferred. These groups include, for example, methylene —CH2-. Ethylidene —CH2CH2-, etc. The term “alkenyl” means an aliphatic hydrocarbon group having at least one carbon-carbon double bond and which may be straight or branched and comprises about 2 to 10 carbon atoms. The alkenyl groups may be substituted with one or more substituents. The term “alkynyl” means an aliphatic hydrocarbon group containing one or more carbon-carbon triple bonds and said groups can be 2 to 10 carbon atoms, with 2 to 6 being most preferred. “Aryl” means an aromatic monocyclic or multicyclic ring system comprising about 6 to 14 carbon atoms, preferably about 6 to 10. Phenyl is a preferred aryl group. The term “heteroaryl” means an aromatic monocyclic or multicyclic ring system comprising about 5 to 14 ring atoms, preferably about 5 to about 10 and in which one or more ring atoms is an element other than carbon such as a heteroatom selected from nitrogen, sulfur or oxygen. The aryl or heteroaryl groups may be substituted with one or more typical substituents for these systems. These can include, without limitation, halogen, alkyl (unsubstituted or substituted), amino, hydroxyl, etc. Heteroaryl groups may be selected from, for example, pyridyl, pyrazinyl, furanyl, thienyl, pyrimidinyl, pyridine, isoxazolyl, isothiazolyl, oxazolyl, thiazolyl, 1,2, 4-thiadiazolyl, pyrazinyl, pyridazinyl, quinoxalinyl, phthalazinyl, oxindolyl, inidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoaaindolyl, 1,2,4-triazinyl, benzothiazolyl, tetrazolyl and/or dihydro and/or oxo and/or partially saturated versions of such compounds. The term “arylalkyl” or “alkylaryl” or “heterocycloalkyl” means a combination of such groups having the combined meaning of the respective separate groups or elements. The term “cycloalkyl” means a non-aromatic mono- or multi-cyclic ring system comprising about 3 to 10 carbon atoms. The term “halogen” means fluorine, chlorine, bromine or iodine. The definitions recited in U.S. Pat. No. 8,026,364 are hereby incorporated by reference. When the structures shown herein do not specify stereochemistry, the structure(s) can include mixtures or any one of the individual stereoisomers. The term “solvate” means a physical association of a compound with one or more solvent molecules including water (hydrate) and other solvents such as ethanol. The term “effective amount” means an amount of a compound that provides a therapeutically effective amount in a patient in need of treatment thereof. Pharmaceutically acceptable salts are included within the scope of the invention and such salts denote or mean acidic and/or basic salts depending upon the particular compound. Such salts may be hydrochloride salts and the like.

The compounds shown in Table I and having the formula shown as (IB) wherein each of R¹ and R² are as shown in Table I are useful in the treatment of pruritus and other diseases recited herein. The preferred compounds useful in such treatment include those compounds having a cyano moiety as R¹ or R².

TABLE I

IB

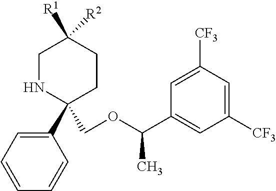

| Com- | | |
|---|---|---|
| pound | R¹ | R² |
| 1 | 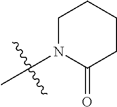 [SMILES: *N1CCCCC1=O] | —CN |
| 2 | 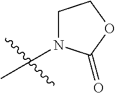 [SMILES: *N1CCOC1=O] | —CN |
| 3 | 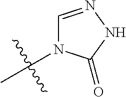 [SMILES: *n1cn[nH]c1=O] | 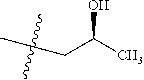 [SMILES: *C[C@H](C)O] |
| 4 | 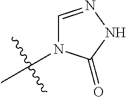 [SMILES: *n1cn[nH]c1=O] | 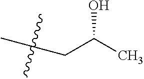 [SMILES: *C[C@@H](C)O] |
| 5 | 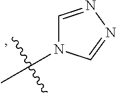 [SMILES: *n1cnnc1] | —CH₂CN |
| 6 | —CH₃ | 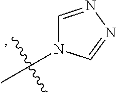 [SMILES: *n1cnnc1] |
| 7 | —CN | 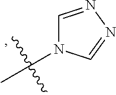 [SMILES: *n1cnnc1] |
| 8 | —C(O)OCH₃ | 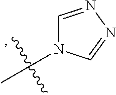 [SMILES: *n1cnnc1] |
| 9 | 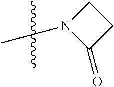 [SMILES: *N1CCC1=O] | —CN |
| 10 | —C(O)NH₂ | 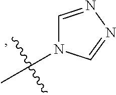 [SMILES: *n1cnnc1] |
| 11 | 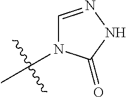 [SMILES: *n1cn[nH]c1=O] | —CH₂C(OH)(CH₃)₂ |
| 12 | —CH₂OH | 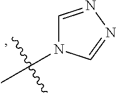 [SMILES: *n1cnnc1] |
| 13 | 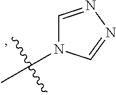 [SMILES: *n1cnnc1] | —CH₂OCH₃ |
| 14 | —CH₂OCH₃ | 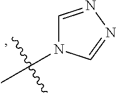 [SMILES: *n1cnnc1] |
| 15 | 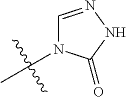 [SMILES: *n1cn[nH]c1=O] | —CH₂NH—S(O₂)CH₃ |
| 16 | 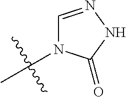 [SMILES: *n1cn[nH]c1=O] | —CH₂C(O)NH(CH₂CH₃) |
| 17 | 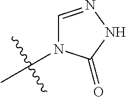 [SMILES: *n1cn[nH]c1=O] | —CH₂C(O)OCH₂CH₃ |
| 18 | 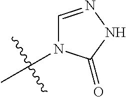 [SMILES: *n1cn[nH]c1=O] | —C(═N—OH)—CH₂CH₃ |
| 19 | 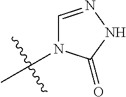 [SMILES: *n1cn[nH]c1=O] | —C(O)CH₂CH₃ |
| 20 | 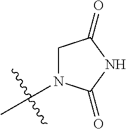 [SMILES: *N1CC(=O)NC1=O] | —CH₂OCH₃ |
| 21 | 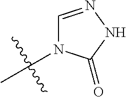 [SMILES: *n1cn[nH]c1=O] | —C(O)NH(CH₃) |
| 22 | —C(O)NH(CH₃) | 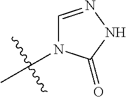 [SMILES: *n1cn[nH]c1=O] |
| 23 | 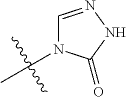 [SMILES: *n1cn[nH]c1=O] | —CH₂OH |
| 24 | 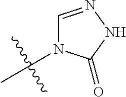 [SMILES: *n1cn[nH]c1=O] | —CH₂CH₂OH |
| 25 | 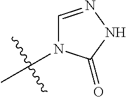 [SMILES: *n1cn[nH]c1=O] | —CH₂CH₂CH₃ |
| 26 | 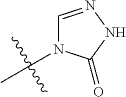 [SMILES: *n1cn[nH]c1=O] | —CH₂OCH₃ |
| 27 | —CH₂OCH₃ | 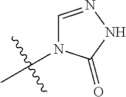 [SMILES: *n1cn[nH]c1=O] |
| 28 | 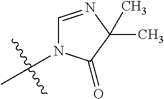 [SMILES: *N1C=NC(C)(C)C1=O] | H |
| 29 | 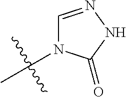 [SMILES: *n1cn[nH]c1=O] | —CH₂C(O)NH₂ |
| 30 | 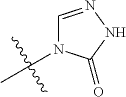 [SMILES: *n1cn[nH]c1=O] | —CH₂C(O)CH₃ |
| 31 | 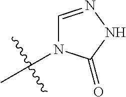 [SMILES: *n1cn[nH]c1=O] | —CH₂C(O)OCH₃ |
| 32 | 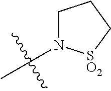 [SMILES: *N1CCCS1(=O)=O] | —CN |
| 33 | 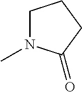 | —CN |
| 34 | —CN | 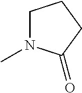 |
| 35 | —CN | 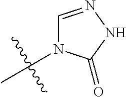 [SMILES: *n1cn[nH]c1=O] |
| 36 | —NHS(O₂)CH₃ | —CN |
| 37 | —CN | —NHS(O₂)CH₃ |
| 38 | 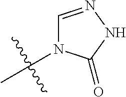 [SMILES: *n1cn[nH]c1=O] | —CH₂CN |
| 39 | —CN | —NH₂ |
| 40 | —NH₂ | —CN |
| 41 | —NHC(O)NH₂ | —CN |
| 42 | 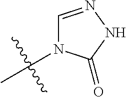 [SMILES: *n1cn[nH]c1=O] | —CN |
| 43 | 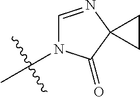 [SMILES: *N1C=NC2(CC2)C1=O] | H |
| 44 | 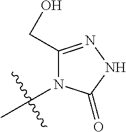 [SMILES: *n1c(CO)n[nH]c1=O] | H |
| 45 | 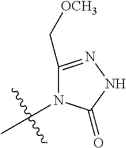 [SMILES: *n1c(COC)n[nH]c1=O] | H |
| 46 | H | 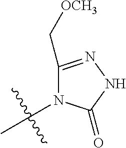 [SMILES: *n1c(COC)n[nH]c1=O] |
| 47 | 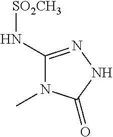 | H |
| 48 | H | 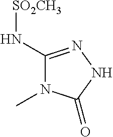 |
| 49 | 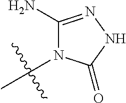 [SMILES: *n1c(N)n[nH]c1=O] | H |
| 50 | H | 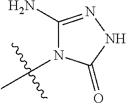 [SMILES: *n1c(N)n[nH]c1=O] |
| 51 | 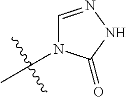 [SMILES: *n1cn[nH]c1=O] | —C(O)NH₂ |
| 52 | 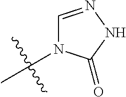 [SMILES: *n1cn[nH]c1=O] | —C(═N—OCH₃)—CH₃ |
| 53 | 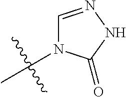 [SMILES: *n1cn[nH]c1=O] | —C(O)CH₃ |
| 54 | 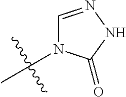 [SMILES: *n1cn[nH]c1=O] | —C(═N—OH)—CH₃ |
| 55 | 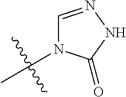 [SMILES: *n1cn[nH]c1=O] | —C(O)OCH₃ |
| 56 | 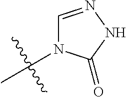 [SMILES: *n1cn[nH]c1=O] | —CH₂C¹ |
| 57 | 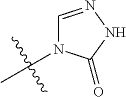 [SMILES: *n1cn[nH]c1=O] | —CH₃ |
| 58 | 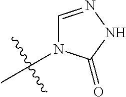 [SMILES: *n1cn[nH]c1=O] | —C(═N—OCH₃)—CH₂CH₃ |
| 59 | —NHC(O)CH₃ | 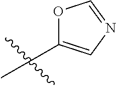 [SMILES: *c1cnco1] |
| 60 | H | 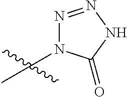 [SMILES: *n1nn[nH]c1=O] |
| 61 | 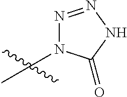 [SMILES: *n1nn[nH]c1=O] | H |

The present invention is directed to the use of the compounds shown in Table I and their pharmaceutically acceptable salts thereof in the treatment of a disease or condition selected from pruritus or prurigo nodularis. The present invention is directed to the use in the manufacture of a medicament of such compounds for such treatment. The compounds of formula I and the formulae shown herein are made by synthetic processes as described in U.S. Pat. No. 7,709,641, said processes hereby incorporated by reference. Key intermediates are produced as described in U.S. Pat. No. 7,049,320 which is hereby incorporated by reference. Therein it is shown that a compound having formula A3 (with the variables as defined therein) undergoes a Wittig reaction to form A57 which is hydrogenated to form A58 which is cyclized to form A59 which is hydroxylated to form A50 which is oxidized to form A61. A61 then undergoes the reactions set forth in U.S. Pat. No. 7,709,641 to produce the compounds disclosed therein including, for example, compounds 2, 9, 10, 12, 14, 15, 19, 20, 23, 29, 30, 42 and 54. Improvements to such processes are described herein. The in vitro and in vivo NK1 activity of the compounds of formula I and others as shown herein can be determined by procedures known in the art. Such data is disclosed in the 7,709,641 patent which is hereby incorporated by reference. This data can be used in combination with data generated to test for therapeutic efficacy for pruritus and/or prurigo nodularis in patients enrolled in a clinical trial for such disease or condition to inform the medical practitioner. The compound known as rolapitant may also be used to treat pruritus and/or prurigo nodularis. The compounds of the invention exhibit potent affinities for the NK1 receptor as measured by Ki values (in nM). The activity or potency for the compounds are determined, in part, by measuring their Ki values and, in part, by testing their efficacy in well controlled clinical trials. The NK1 average Ki values for compounds of the formula shown herein generally range from about 0.01 nM to about 1000 nM in activity. Compounds shown as 2, 9, 10, 12, 14, 15, 19, 20, 23, 29, 30, 42, and 54 (see Table I) have Ki values respectively of 0.12, 0.18, 0.1, 0.05, 0.1, 0.13, 0.1, 0.11, 0.12, 0.11, 0.54, 0.28 and 0.12 nM.

The compounds of the invention useful for treating pruritus and/or prurigo nodularis may be formulated into pharmaceutical compositions comprising a compound of the formula shown herein and a pharmaceutically acceptable excipient. The weight percentage of the active ingredient in combination with the inert excipients depends upon the particular drug and the dosage form or delivery method. Pharmaceutically acceptable excipients may be solid or liquid. Solid form pharmaceuticals may be in the form of powders, tablets, capsules, granules, cachets or other known forms. The present invention thus comprises a pharmaceutical composition for the treatment of pruritus comprising a compound of formula I and other formulae shown herein and a pharmaceutically acceptable excipient.

The quantity of active compound in a unit dosage form may vary from about 0.01 mg to about 500 mgs with a preferred range being 0.04 mgs to about 250 mgs. The preferred dosage form is an oral, once per day dose. The compounds useful in the treatment of pruritus and/or prurigo nodularis may also be used in combination with other active ingredients used in the treatment of severe itching or itching associated with other diseases and conditions. Such active ingredients include antihistamines, steroids, opioids and other first line therapy and, in some cases, antidepressants. The compounds may be used for the treatment of acute and chronic pruritus and is inclusive of drug-induced pruritus, paraneoplastic pruritus, cutaneous T-cell lymphoma associated pruritus, brachioradial pruritus and prurigo nodularis.

The present invention further comprises:

A pharmaceutical composition comprising:
a) a compound of Formula II

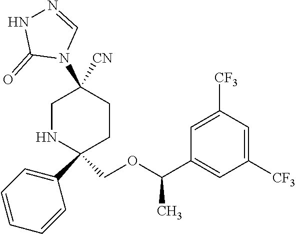 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable stereoisomer or salt thereof and, optionally,
b) a pharmaceutically acceptable vehicle selected from the group consisting of a diluent, binder, disintegrant, wetting agent or lubricant.

The invention also comprises a composition according to the above formulation in an oral dosage form;

a composition according to the above formulation having 2.5 mg to 250 mgs of a compound of formula II in the free amine form;
a composition according to the above formulation in the form of a capsule, tablet or suspension.

The invention comprises a method for treating chronic cough in a patient in need of treatment, the method comprising administering to the patient an effective amount of the pharmaceutical composition described herein with a substantially pure crystalline form of the compound of formula II.

The invention also comprises, a method for treating overactive bladder (OAB) in a patient in need of treatment thereof, the method comprising administering to the patient an effective amount of the pharmaceutical composition having a compound of formula II in substantially pure form or in crystalline form.

The invention further comprises a method for treating depression in a patient in need of treatment thereof, the method comprising administering to the patient an effective amount of the pharmaceutical composition having a compound of formula II and a method for treating alcohol dependency in a patient in need of treatment thereof, the method comprising administering to the patient an effective amount of a composition comprising a compound of formula II, such methods comprising the use of a substantially pure form or a crystalline form of a compound of formula II.

In a preferred embodiment, the invention comprises a method for treating pruritus in a patient in need of treatment thereof, the method comprising administering to the patient an effective amount of a pharmaceutical composition comprising a compound of formula II and a method for treating prurigo nodularis in a patient in need of treatment thereof, the method comprising administering to the patient an effective amount of a pharmaceutical composition comprising a compound of formula II.

The invention further comprises a combination comprising a pharmaceutically effective amount of a formulation according to claim 1 and at least one additional active pharmaceutical ingredient selected from the group consisting of an antidepressant, an anti-nausea or anti-emetic medication, a chemotherapeutic agent or an anti-inflammatory agent.

The invention also comprises a combination wherein the antidepressant is selected from an SSRI.

The invention also comprises a combination wherein the anti-nausea/anti-emetic medication is selected from a 5-HT3 receptor antagonist.

The invention further comprises a crystalline form of a compound having the formula:

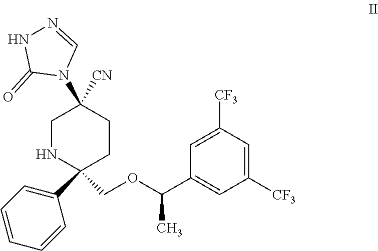 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable salt thereof.

The invention comprises a crystalline form of formula II as the free amine and which is non-hygroscopic.

The invention comprises a crystalline form of formula II in the form of a powder.

The invention comprises a pharmaceutical composition comprising a compound of formula II in crystalline form and a pharmaceutically acceptable excipient.

The invention comprises a pharmaceutical composition comprising a crystalline compound of formula II and a pharmaceutically acceptable excipient wherein the excipients are selected from the group consisting of a diluent, binder, disintegrant, wetting agent or lubricant.

The invention comprises an amorphous form of a compound of formula II or a pharmaceutically acceptable salt thereof.

The invention comprises an amorphous salt form of a compound of formula II wherein the salt is a hydrochloride salt or a tosylate salt.

The invention comprises a method of treating acute and delayed nausea and vomiting associated with initial and repeat courses of highly emetogenic cancer chemotherapy comprising administration of an intravenous formulation of a compound of formula II in combination with at least one additional antiemetic agent.

The invention comprises a method of treating acute and delayed nausea and vomiting associated with initial and repeat courses of moderately emetogenic cancer chemotherapy comprising administration of an intravenous formulation of a compound of formula II or crystalline form thereof in optional combination with at least one additional antiemetic agent.

The invention comprises a method of treating emesis in a patient in need of treatment thereof comprising administration of a pharmaceutically effective amount of a crystalline compound of formula II.

The invention comprises administering a bolus dose of a compound of formula II in a pharmaceutical composition as recited herein.

The invention comprises a compound of Formula IIa, IIb and IIc and pharmaceutically acceptable salts thereof:

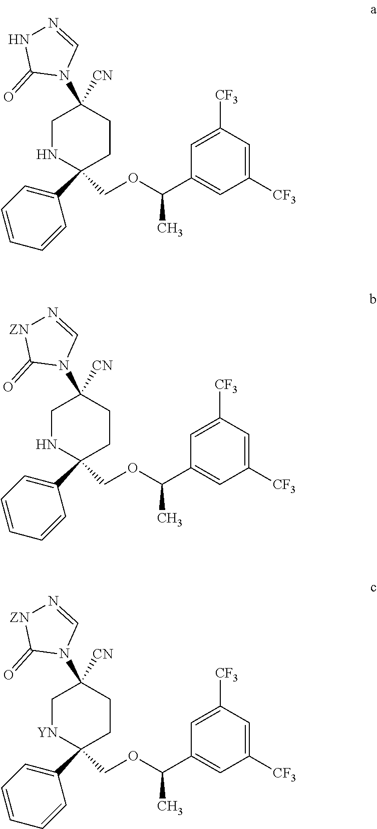

wherein Z and Y are independently selected from the group consisting of —PO(OH)O-M+, —PO(O-)22M+, —PO(O-)₂D₂+, —[C(R¹)(R²)]n-PO(OH)O-M+, —[C(R¹)(R²)]n-PO(O-)22M+, —[C(R¹)(R²)]n-PO(O-)₂D₂+, —C(O)[C(R¹)(R²)]m-OPO(O-)22M+, —C(O)[C(R¹)(R²)]oNR¹R², —C(O)[C(R¹)(R²)]pCO₂-M+, —SO₃-M+, —[C(R¹)(R²)]qSO₃-M+ and —[C(R¹)(R²)]rOC(O)OR³, wherein R³ is selected from the group consisting of

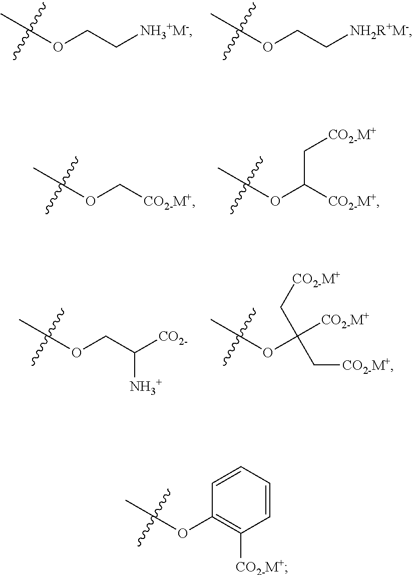

M+ is selected from a monovalent cation; D+ is selected from a divalent cation; R¹ and R² are independently selected from H or C₁₋₆ alkyl; n is 1-4; m, o, and p are independently selected from 0-4; and R is selected from C₁₋₆ alkyl.

The invention comprises a compound of formula IIa, IIb or IIc wherein Z is selected from H and Y is selected from any one of the groups shown above for Z and Y exclusive of H.

The invention comprises a compound of formula IIa, IIb or IIc wherein M⁺ is selected from ammonium salts, alkali metal salts such as sodium, alkaline earth metal salts such as calcium and magnesium, salts with organic bases such as N-methyl-D-glucamine or dicyclohexylamine or amino acid salts such as arginine or lysine.

The invention also comprises a method of treating acute and delayed nausea and vomiting associated with initial and repeat courses of highly emetogenic cancer chemotherapy comprising administration of an intravenous formulation of a compound of formula IIa, IIb or IIc in combination with at least one additional antiemetic agent.

The present invention comprises a method of treating acute and delayed nausea and vomiting associated with initial and repeat courses of moderately emetogenic cancer chemotherapy comprising administration of an intravenous formulation of a compound of formula IIa, IIb or IIc in combination with at least one additional antiemetic agent.

The invention comprises an intravenous formulation comprising a compound of formula IIa, IIb or IIc.

The invention comprises a method of treating a patient in need of treatment thereof for CINV comprising administering an intravenous formulation having a compound of formula II wherein the formulation is administered at one dose per chemotherapeutic treatment cycle.

The invention comprises a pharmaceutical formulation comprising a compound of formula II or a pharmaceutically acceptable salt thereof wherein the T½ for the active ingredient is about 10-13 days and wherein upon administration the blood concentration (ng/mL) for doses ranging from about 50 to about 200 mg and directly after administration ranges from about 280 ng/mL to about 850 ng/mL.

The invention comprises a micelle formulation suitable for intravenous administration comprising a compound of formula II or pharmaceutically acceptable salts thereof.

The invention comprises an emulsion formulation suitable for intravenous administration comprising a compound of formula II or a pharmaceutically acceptable salt thereof.

The invention comprises a pharmaceutical composition comprising a compound of formula II or pharmaceutically acceptable salts thereof and a non-ionic solubilizer.

The invention comprises a pharmaceutical formulation comprising a compound of formula II and having stability for up to eighteen months when stored at a temperature range of 15-30 degrees C.

The invention comprises an aqueous suspension comprising a compound of formula II and a cellulosic polymer.

The invention comprises a suspension comprising a compound of formula II and a cellulosic polymer wherein the polymer is selected from the group consisting of hydroxypropylmethylcellulose.

A compound of formula I (also designated SCH 900978) is a potent, selective, competitive neurokinin-1 (NK-1) receptor antagonist that is effective as an oral administered drug for the treatment of cough and meets this unmet medical need. In addition, this drug is effective for the treatment of overactive bladder (OAB) and other selected indications including major depression and alcohol dependency. This compound binds with high affinity to the human NK1 receptor and comprises a compound having an equilibrium dissociation constant determined by radio ligand binding of [Ki] equal to 0.28 nM (human). The present invention further has been found to competitively antagonize functional effects mediated by activation of the NK1 receptor in cultured cells with an equilibrium dissociation constant determined by Schild analysis [Kb] of 0.17 nM. The present invention comprises a compound of formula I having an effective dose for 90% inhibition [ED90]=0.2 mg/kg as determined in an in vivo pharmacodynamic model of NK1 receptor activity (gerbil foot thumping). The present inventors have found that the compound of formula I is selective for NK1 and has low affinity for NK2 and NK3 receptors (Ki>1 uM). The present invention relates to a method of treating chronic pruritus in a patient in need of treatment thereof comprising administering a pharmaceutically effective amount of a compound of formula II having a Ki equal to about 0.28 nM; a Kb of 0.17 nM; an effective dose [ED90] of 0.2 mg/kg and having low affinity (Ki>1 uM) for NK2 and NK3 receptors and high affinity for NK1 receptor.

The present invention comprises a compound having activity in mammalian cough models and is thus useful in the treatment of cough in mammals, including humans, after oral administration. The present invention comprises an oral formulation of a compound of formula I and pharmaceutically acceptable excipients selected from the group consisting of diluents, binders, disintegrants, wetting agents and lubricants. In a preferred embodiment the pharmaceutical composition comprises a compound of formula I (or IIa-IIc) or II and lactose monohydrate, microcrystalline cellulose, povidone, crospovidone, poloxamer 188 and magnesium stearate. The preferred oral form is a capsule. The amount of active ingredient in the capsule or oral dosage form may range from 1 mg to 250 mgs with a preferred range of 10-150 mg with a more preferred range of 20-100 mg per day in humans. The present invention also relates to an NK-1 antagonist (e.g., Formula II) having a half-life of approximately 10-13 days. The present invention further relates to a method of treating pruritus comprising administering a pharmaceutically effective amount of a compound of formula II having a half-life of 10-13 days to a patient in need of treatment thereof. In a preferred embodiment, the present invention relates to a pharmaceutical composition comprising a compound of formula I and wherein said compound has a half-life upon administration to a patient in need of treatment thereof of approximately 10-13 days. The preferred form of a compound of formula I is as the free amine and in non-hygroscopic, crystalline free base form (preferably as a crystalline powder). SCH900978 has a calculated pKa at 1.8, 7.0 and 7.6. The solubility of this drug at pH 7 is 1.8 ug/mL. The log D at pH 7.4 in an octanol/water system is 4.5. The measured onset melting temperature as measured by differential scanning calorimetry (DSC) is 168.9. The molecular weight is 539.5.

Prodrugs of a compound of formula I or II may also be utilized in the formulations suitable for oral or parenteral administration. Prodrugs wherein either free amine (or both amines) in a compound of formula I or II has the hydrogen on the piperidine ring (and or other nitrogen ring) replaced with a group selected from —Y and/or —Z and salts thereof wherein Y and/or Z is selected from —P(O)(OH)₂, —S(O)n1R¹, —C(O)(C₁₋₆ alkyl)X, —C(O)(C₁₋₆ alkyl)(aryl), —C(O)OR⁴; X is selected from —NR²R³, —P(O)(OH)₂ or —S(O)n1R¹; R¹ is H or C₁₋₆ alkyl; R² is H or C₁₋₆ alkyl; R³ is H or C₁₋₆ alkyl; R⁴ is H or C₁₋₆ alkyl; n1 is 0-4 (formula IIa or IIb or IIc) are suitable for use herein. Suitable cations or di-cations for the ionized form(s) of the prodrugs include metal salts or organic amine cations including meglumine salts and the like (N-methyl D-glucamine). Such prodrugs may be utilized with or without the described parenteral delivery vehicles in a suitable liquid formulation to treat patients in need of treatment thereof. Such prodrugs are converted to the non-prodrug form of the drug (or salt thereof) upon parenteral administration to the patient. Such prodrugs may be in amorphous form or in crystalline and/or crystalline solvate/hydrate form.

NK-1 receptor antagonists have been shown to be useful therapeutic agents, for example, in the treatment of pain, inflammation, migraine, nausea, emesis (vomiting), and nociception. The compound of formula I is believed to be particularly useful in treating cough, overactive bladder (OAB) and central nervous system disorders and diseases such as depression. The US patents referenced above and incorporated by reference herein generally describe multiple indications for the use of said NK-1 antagonists of the generic and specific compounds disclosed therein. In particular, such indications include cough as well as other disorders including central nervous system disorders such as depression. There is no specific recitation of a formulation of a compound of formula I nor a particular form or salt form thereof in the prior art. The present invention relates to a specific formulation of this NK-1 antagonist and pro-drugs thereof and to other embodiments as described herein including specific salt forms and crystalline and/or amorphous forms of a compound of formula I.

The present invention broadly relates to formulations suitable for administration to a patient in need of treatment thereof wherein said formulations comprise a compound of Formula I or II and pharmaceutically acceptable salts, hydrates, solvates thereof and, (i) in an oral formulation, pharmaceutically acceptable excipients comprising a diluent, a binder, a disintegrant, a wetting agent and a lubricant or (ii) in a parenteral formulation, additional excipients include a vehicle selected from the group consisting of water soluble organic solvents, non-ionic surfactants, water insoluble lipids, organic lipids/semisolids and phospholipids. Water soluble organic solvents may be selected from, for example, polyethylene glycol 300, polyethylene glycol 400, ethanol, propylene glycol, glycerin, N-methyl-2-pyrrolidone, dimethylacetamide and dimethylsulfoxide.

Non-ionic surfactants may be selected from Cremophor EL, Cremophor RH 40, Cremophor RH 60, d-α-tocopherol polyethylene glycol 1000 succinate, polysorbate 80, Solutol HS 15, sorbitan monooleate, poloxamer 407, Labrifil M-1944CS, Labrafil M-2125CS, Labrasol, Gellucire 44/14, Softigen 767, and mono- and di-fatty acid esters of PEG 300, 400 or 1750. The water insoluble lipids are selected from castor oil, corn oil, cottonseed oil, olive oil, peanut oil, peppermint oil, safflower oil, sesame oil, soybean oil, hydrogenated vegetable oils, hydrogenated soybean oil, and medium chain triglycerides of coconut oil and palm seed oil. Organic liquids and semisolids may be selected from beeswax, d-α-tocopherol, oleic acid and medium chain mono- and diglycerides. The phospholipids are selected from lecithin, hydrogenated soy phosphatidylcholine, distearoylphosphatidylglycerol, L-α-dimyristoylphosphatidylcholine and L-α-dimyristoylphosphatidylglycerol and others as disclosed herein. The intravenous formulations are made to provide sufficient solubility and chemical stability which is defined as <5-10% degradation over one year (preferrably two years) under the specified storage conditions which vary depending upon the particular formulation, location etc.

Preferably, the formulations are useful as oral formulations. As necessary or prescribed, the formulations may also be used broadly as parenteral formulations suitable for delivery by means known in the art including intravenous (IV), intramuscular (IM) or subcutaneous (SC) administration. The prodrugs may be used in oral form or in parenteral formulations comprising an aqueous/saline delivery system with or without the optional delivery vehicles recited above. The oral formulation is preferably in the form of a capsule and specifically includes, for example, lactose monohydrate and microcrystalline cellulose as the diluents; povidone as a binder; crospovidone as a disintegrant; poloxamer 188 as a wetting agent and mangnesium stearate as a lubricant.

The present invention describes and claims inter alia pharmaceutical compositions and formulations of Formula I or II and pharmaceutically acceptable salts thereof for use in treating nausea and/or emesis and other NK-1 related diseases and conditions. These diseases include chronic cough, depression, alcohol dependency and overactive bladder (OAB) and a host of other diseases and conditions which benefit from selective inhibition of the NK-1 receptor. In addition, such compounds are useful for the treatment of pruritus (chronic and acute) and prurigo nodularis. The preferred form of a compound of formula II used to formulate the compositions recited herein, including the iv formulations, is as the free amine crystalline form. Pharmaceutically acceptable salts including hydrochloride or tosylate salts or other salt forms (amorphous or crystalline) or hydrates or solvates thereof may also be prepared. The term “crystalline free form” means the crystalline, free amine form of the drug or crystalline, free amine form of a compound of formula I or II. The crystalline form may be in a crystalline, powder form.

The present invention relates to formulations comprising,
a) a compound of formula II or a pharmaceutically acceptable salt thereof,

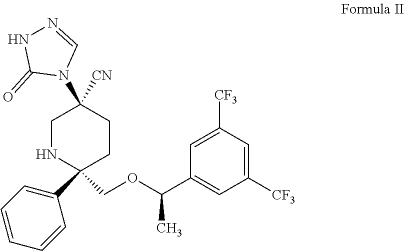 [SMILES: C[C@@H](OC[C@@]1(c2ccccc2)CC[C@](C#N)(n2cn[nH]c2=O)CN1)c1cc(C(F)(F)F)cc(C(F)(F)F)c1]

and
b) pharmaceutically acceptable excipients selected from the group comprising a diluent, a binder, a disintegrant, a wetting agent and a lubricant or (ii) in a parenteral or iv formulation, additional excipients include a pharmaceutically acceptable vehicle selected from the group consisting of water soluble organic solvents, non-ionic surfactants, water insoluble lipids, organic lipids/semisolids and phospholipids.

The term “pharmaceutically acceptable vehicle” means any suitable component which enhances the solubility of a compound of formula I or a pharmaceutically acceptable salt thereof in order to facilitate the parenteral delivery of a therapeutic concentration of such compound or salt to the target NK-1 receptor site(s). The vehicles are selected from the group consisting of cremophors, emulsions, microemulsions, micelles, negatively charged micelles, oil loaded micelles, intralipids, HSA, liposomes and negatively and positively charged amino acids and the like as additionally described herein. In the case of liposomes, emulsions, micelle and oil-loaded micelles, it is believed that such vehicles would hold the drug inside the lipophilic core for enhanced drug retention while also shielding the drug in the core. Human Serum albumin based formulations relate to the strong binding of HSA to Compound 1 (Formula II) and which would minimize the partitioning of free drug into red blood cells. Such formulations may be co-formulated with Solutol, Myglyol and vitamin E. Negatively charged amino acids would complex with and neutralize a portion of Compound 1 (Formula II) that retains a positive charge and thus prevent the partitioning of Compound 1 into red blood cells. The positively charged amino acids would complex with a negatively charged portion of Compound 1 and neutralize it and reduce exposure of the compound to red blood cells. A negatively charged micelle would repel the negatively charged erythrocytes and prevent contact of Compound 1 with red blood cells.

The term “pharmaceutically acceptable excipient” means those pharmaceutical excipients specifically exemplified herein and those within the same class of excipients specifically used herein and including disintegrants, binders, lubricants, wetting agents and diluents known in the art.

The term “micelle formulation” means that the formulation is in the form of a micelle and is derived from or made up of any component which forms or can form a micelle in a pharmaceutically acceptable delivery system such as water, saline, dextrose water, and the like.

The term “emulsion formulation” means that the formulation is in the form of an emulsion and which is derived from or made up of any component which forms or can form an emulsion when presented in and/or combined with a pharmaceutically acceptable delivery system such as water, saline, dextrose water and the like. The preferred emulsion formulations which avoid any hemolytic effects upon bolus or slow infusion administration have an oil content of about 10% or less. The drug concentration can be varied from about 1 mg/mL to about 30 mg/mL with less volume and higher concentration being preferred for intravenous delivery. The pharmaceutical compositions can be prepared to increase or enhance the solubility of the NK-1 antagonist and can also be diluted significantly to avoid any possible hemolytic results but some dilution volumes may be impractical to administer to a patient in need of treatment thereof.

Abbreviations, Acronyms, terms or units have the following definitions:
ACN Acetonitrile
AUC Area under the plasma concentration curve
AUC(0-x hr) Area under the curve of plasma concentration-time curve from time zero to x hours after dosing
AUC(l) Area under the plasma concentration-time curve from time 0 to infinity
AUC(tf) Area under the plasma concentration-time curve from time 0 to time of final quantifiable sample
Cmax Maximum observed plasma concentration
CNS Central nervous system
CP Chronic pruritus
CV Coefficient of variation
CYP Cytochrome P450
Da Dalton
DBP Diastolic blood pressure
DSC Differential scanning calorimetry
ECG Electrocardiogram
ED90 Effective dose for 90% inhibition
EFD Embryofetal development
EM Exposure multiple
F Female
FEED Fertility and early embryonic development
GLP Good laboratory practice
HDPE High-density polyethylene
hERG human ether-a-go-go-related gene
HR heart rate
IC50 concentration at which inhibition is one-half maximal
IP Intraperitoneal
Kb Equilibrium dissociation constant determined by Schild analysis
Ki Equilibrium dissociation constant determined by radioligand binding
LC liquid chromatography
LC-MS/MS liquid chromatographic tandem mass spectrometric
LLOQ lower limit of quantitation
M Male
MBP mean blood pressure
mpk milligram per kilogram
mRNA messenger ribonucleic acid
MV Minute volume
n number
ND not determined
NK-1 nuerokinin-1
NOAEL no observed adverse effect level
NTS nucleus tractus solitaries
OAB overactive bladder
PET positron emission tomography
P-gp P-glycoprotein
PMC pontine micturition center
PO per os
PXR pregnane X receptor
RR respiratory rate
SBP systolic blood pressure
T½ apparent terminal elimination phase half-life
tf time of final quantifiable sample
Tmax time to maxiumum observed plasma concentration
TV tidal volume
w/v weight/volume

An oral formulation or emulsion formulation or micelle formulation of the present invention comprises an active pharmaceutical ingredient selected from a compound of formula I, II or IIa or IIb or IIc (shown below as A, B and C) with Z and Y as defined above and/or pharmaceutically acceptable salts, hydrates, polymorphs or physical forms thereof.

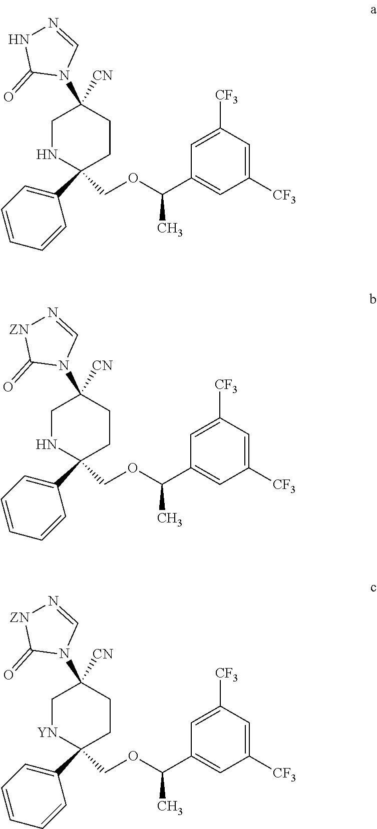

Such drug loaded emulsion or micelle formulations may additionally contain excipients which facilitate delivery and/or are useful to prevent or mitigate factors such as hemolysis. Such additional excipients can thus include, for example, oils or other components which enhance or further enhance solubility while mitigating any potential hemolytic effects.

Such emulsion formulations or micelle formulations may be further processed to form more stable physical forms or solutions and can be further processed to, for example, provide sterilized parenteral solutions.

The present invention also relates to oral or parenteral formulations comprising a compound of formula I or II (or IIa or IIb or IIc) or a pharmaceutically acceptable salt thereof in the form of a nanoparticle. The nanoparticles of a compound of formula I or salt thereof may then be incorporated in a solution to deliver such nanoparticle by intravenous means. The nanoparticles of a compound of formula II and pharmaceutically acceptable salts thereof may further include a pharmaceutically acceptable vehicle. It is believed that slow dissolution of such nanoparticles (˜200 nm) would result in less hemolysis due to less dissolved drug in a solubilized fraction.

The present invention also relates to a method of delivering a compound of formula I, II, IIa, IIb, or IIc and pharmaceutically acceptable salts thereof to a patient comprising (a) combining a compound of formula II or pharmaceutically acceptable salt thereof with a pharmaceutically acceptable vehicle to form an intravenous formulation (b) delivering the parenteral formulation to a patient in need of treatment thereof. In the case of an oral formulation, tablets or capsules may be prepared with the capsule being the preferred oral form.

In one aspect, the present invention relates to a pharmaceutical composition suitable for parenteral administration, comprising:
a) a compound of formula II or pharmaceutically acceptable salt thereof

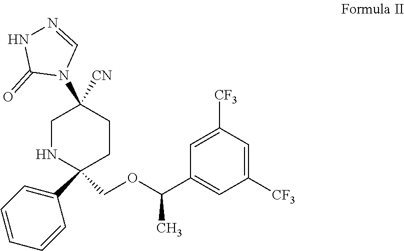 [SMILES: C[C@@H](OC[C@@]1(c2ccccc2)CC[C@](C#N)(n2cn[nH]c2=O)CN1)c1cc(C(F)(F)F)cc(C(F)(F)F)c1]

b) a solubilizer selected from the group consisting of an oil-loaded micelle or a microemulsion.

Another embodiment of the invention comprises a pharmaceutical intravenous formulation which comprises:
a) a compound of Formula II or a pharmaceutically acceptable salt thereof

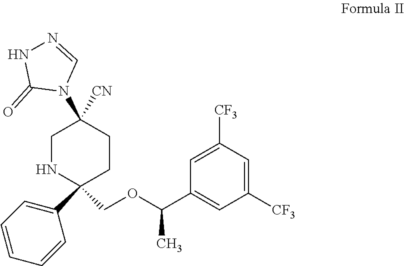 [SMILES: C[C@@H](OC[C@@]1(c2ccccc2)CC[C@](C#N)(n2cn[nH]c2=O)CN1)c1cc(C(F)(F)F)cc(C(F)(F)F)c1]

and
b) an emulsifier.

The invention also comprises an intravenous formulation comprising a compound of Formula II or a pharmaceutically acceptable salt thereof and human serum albumin (HSA).

The invention also comprises an intravenous formulation comprising a compound of Formula II or a pharmaceutically acceptable salt thereof wherein the compound or salt thereof is in the form of a nanoparticle or micronized particle.

The invention additionally comprises an intravenous formulation comprising a compound of Formula II or a pharmaceutically acceptable salt thereof and a delivery vehicle selected from Cremophor.

The invention further comprises an intravenous formulation comprising a compound of Formula II or a pharmaceutically acceptable salt thereof and a delivery vehicle selected from a micelle.

The invention further comprises an intravenous formulation comprising a compound of Formula II or a pharmaceutically acceptable salt thereof and a delivery vehicle selected from a liposome.

The invention preferably relates to an intravenous emulsion formulation which is suitable for both bolus and infusion administration. Each of the delivery vehicles recited above may also be used for formulas IIa-IIc.

An embodiment of the invention comprises an intravenous formulation comprising a compound of Formula II or a pharmaceutically acceptable salt thereof and at least one emulsifier wherein an emulsion is formed and subject to microfluidization to form droplets having less than 500 nm median diameters and/or a D90 of about 600 nm or less.

The invention further relates to an intravenous formulation comprising a compound of Formula II or a pharmaceutically acceptable salt thereof and a negatively or positively charged amino acid.

The invention further comprises an intravenous formulation comprising a compound of Formula II or a pharmaceutically acceptable salt thereof which is lyophilized.

The invention further comprises an intravenous formulation comprising a compound of Formula II or a pharmaceutically acceptable salt thereof in the form of a powder. The powder is reconstituted or added to a liquid to form a liquid intravenous formulation comprising a compound of Formula II or salt thereof which is administered to a patient in need of treatment thereof. Emulsifiers such as Polysorbate 80 (Tween 80) and the like may be added to this formulation as well as other inactive ingredients such as pH adjusters, preservatives (EDTA) etc.

In each of the above embodiments, the preferred form of the compound of Formula II or a salt thereof added to the formulation is as the solid crystalline free amine form.

In each of the above embodiments, an alternative form of the compound of formula II or a salt thereof is selected from a pro-drug of a compound of formula II. Such pro-drugs may be administered by any delivery means including via an oral route or by intravenous administration.

Such pro-drug may be selected from a compound of Formula IIa, IIb or IIc and pharmaceutically acceptable salts thereof:

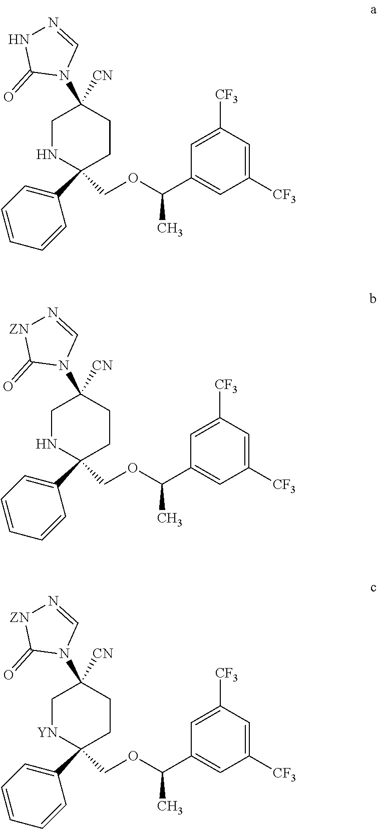

wherein Z and Y are independently selected from the group consisting of —PO(OH)O⁻M⁺, —PO(O⁻)22M⁺, —PO(O⁻)₂D²⁺, —[C(R¹)(R²)]ₙ—PO(OH)O⁻M⁺, —[C(R¹)(R²)]ₙ—PO(O⁻)22M⁺, —[C(R¹)(R²)]ₙ—PO(O⁻)₂D²⁺, —C(O)[C(R¹)(R²)]ₘ—OPO(O⁻)22M⁺, —C(O)[C(R¹)(R²)]ₒNR¹R², —C(O)[C(R¹)(R²)]ₚCO₂-M⁺, —SO₃-M⁺, —[C(R¹)(R²)]_{q}SO₃-M+ and —[C(R¹)(R²)]ᵣOC(O)OR³, wherein R³ is selected from the group consisting of

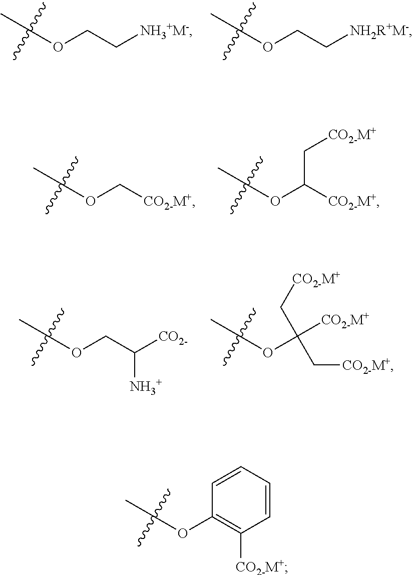

M⁺ is selected from a monovalent cation; D⁺ is selected from a divalent cation; R¹ and R² are independently selected from H or C₁₋₆ alkyl; n is 1-4; m, o, and p are independently selected from 0-4; and R is selected from C₁₋₆ alkyl.

In a more preferred embodiment, such pro-drugs are selected from:

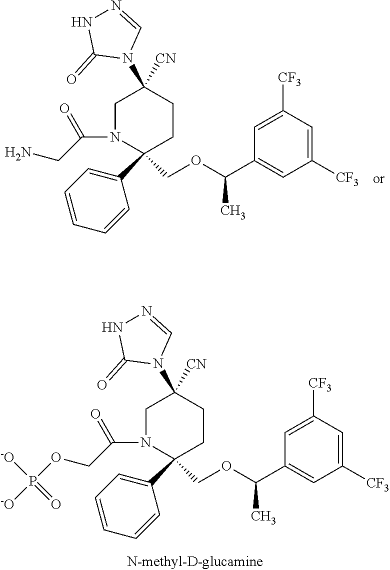

The preferred M+ salts are selected from, for example, ammonium salts, alkali metal salts such as sodium, alkaline earth metal salts such as calcium and magnesium, salts with organic bases such as N-methyl-D-glucamine or dicyclohexylamine, amino acid salts such as arginine, lysine and the like.

The pro-drugs are made by reacting the amine or suitably protected amine with an activated group Z—X or Y—X or by any conventional means to form the pro-drug variant of a compound of formula I or II.

The pro-drugs of the instant invention possess enhanced solubility over the parent drug and are thus useful and suitable for intravenous administration.

In another embodiment, the invention provides a pharmaceutical composition which comprises macrogol 15-hydroxystearate in an amount of from about 0.50% to about 7.5% by weight of the total composition; a medium chain triglyceride in an amount of from about 0.15% to about 1.5% by weight of the total composition; and a long chain triglyceride in an amount of from about 0.10% to about 1.2% by weight of the total composition.

In another embodiment, the invention provides a pharmaceutical composition which comprises macrogol 15-hydroxystearate in an amount of from about 0.88% to about 4.84% by weight of the total composition; a medium chain triglyceride in an amount of from about 0.20% to about 1.20% by weight of the total composition; and a long chain triglyceride in an amount of from about 0.10% to about 0.75% by weight of the total composition.

In another aspect, the invention provides a process for making a pharmaceutical composition which comprises:
a) heating (i) melted macrogol 15-hydroxystearate, (ii) a medium chain triglyceride and (iii) a long chain triglyceride to form a composition;
b) adding water to the composition to form a microemulsion composition;
c) adding to the microemulsion composition a compound of Formula II

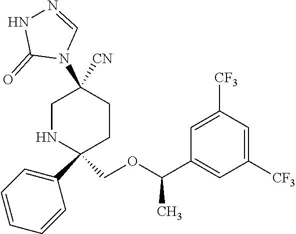 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable salt thereof; and
d) adding at least one buffer and adjusting the pH from about 6.5 to about 8.0 to form a pharmaceutical composition, wherein the macrogol 15-hydroxystearate is present in an amount of from about 0.50% to about 10.0% by weight of the total pharmaceutical composition, the medium chain triglyceride is present in an amount of from about 0.10% to about 2.5% by weight of the total pharmaceutical composition, and the long chain triglyceride is present in an amount of from about 0.10% to about 1.5% by weight of the total pharmaceutical composition, and wherein the weight ratio of macrogol 15-hydroxystearate:medium chain triglyceride:long chain triglyceride in the pharmaceutical composition is about 5-100:1-5:1.

In another aspect, the invention provides a pharmaceutical composition which comprises:
a) a compound of Formula II

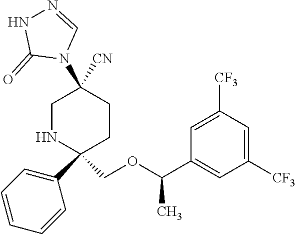 [SMILES: C[C@@H](OC[C@@]1(c2ccccc2)CC[C@](C#N)(n2cn[nH]c2=O)CN1)c1cc(C(F)(F)F)cc(C(F)(F)F)c1]

or a pharmaceutically acceptable salt thereof; and
b) pegylated hydroxystearate in an amount of from about 0.88% to about 5.0% by weight of the total composition, wherein the pegylated hydroxystearate is substantially free from free polyethylene glycol, and wherein the pH of the composition is from about 6.5 to about 8.

In another aspect, the invention provides a method for treating nausea and/or emesis in a patient in need of treatment which comprises intravenously administering by infusion to the patient an effective amount of a pharmaceutical composition of the present invention, wherein hemolysis in the patient is minimized.

In another aspect, the invention provides a method for minimizing hemolysis in a patient following intravenous administration of a compound of the formula II or a pharmaceutically acceptable salt thereof,

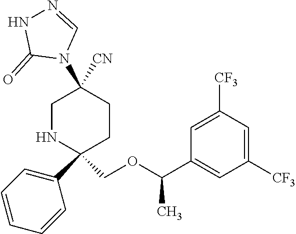 [SMILES: C[C@@H](OC[C@@]1(c2ccccc2)CC[C@](C#N)(n2cn[nH]c2=O)CN1)c1cc(C(F)(F)F)cc(C(F)(F)F)c1]

the method comprising intravenously administering by infusion to the patient an effective amount of a pharmaceutical composition of the present invention.

In another embodiment, the recited oral and/or intravenous formulations may be used in combination with other antiemetic and antinausea medications; anti-inflammatory or steroidal agents (e.g. dexamethasone) and with chemotherapeutic agents. The recited intravenous formulations may be given to the patient according to the prescription and regimen provided by a physician. Such other medications include ondansetron and other known 5HT3 antagonists. Thus, a compound of formula II and salts thereof for injection may be utilized with other antiemetic agents for the prevention of acute and delayed nausea and vomiting associated with initial and repeat courses of highly emetogenic cancer chemotherapy including, for example, treatment with cisplatin. A compound of formula II and salts thereof for injection may also be utilized with other antiemetic agents for the prevention of acute and delayed nausea and vomiting associated with initial and repeat course of moderately emetogenic cancer chemotherapy. In addition to treatment with cisplatin, other anticancer agents that are administered in this combination dosing regimen include etoposide, flurouracil, gemcitabine, vinorelbine, paclitaxel, cyclophosphamide, doxorubicin, docetaxel and can also include temozolomide. The treatment with a compound of formula II should begin thirty minutes before chemotherapy treatment on day 1 of such treatment. The i.v. formulation may be administered by slow infusion over fifteen minutes or by bolus injection depending upon the formulation.

In another embodiment, the i.v. formulations of a compound of Formula II and a pharmaceutically acceptable salt thereof may be administered alone or in combination with other agents for the treatment and/or prevention of post-operative nausea and vomiting. Such combination agents include other antiemetic therapeutic agents such as ondansetron and other 5HT3 antagonists.

The pharmaceutical compositions of the present invention are useful for the treatment of cough. Accordingly, in another aspect, the present invention provides a method for treating cough in a patient in need of treatment, e.g., a human or non-human primate such as a monkey or a companion animal such as a dog or a cat. The method comprises administering to the patient an effective amount of a pharmaceutical composition of the present invention. The pharmaceutical compositions are useful in treating chronic cough. The pharmaceutical compositions are also useful in treating depression and/or anxiety. The pharmaceutical compositions may also be co-formulated to include at least one other antidepressant, e.g., sertraline. The pharmaceutical compositions herein may also be provided in combination with such other anti-nausea and/or emetic agents and/or chemotherapeutic agents as prescribed by a physician.

The pharmaceutical composition, if in liquid form, can be diluted prior to administration with a suitable aqueous diluent(s) such as normal saline, dextrose, dextrose filtered water and mannitol, to obtain any intermediary composition between about 0.88% to about 4.4% Solutol® HS 15 by weight of the total composition.

The dosage regimen utilized for the pharmaceutical composition of the present invention is selected based on consideration of a variety of factors, including species, age, weight, sex and medical condition of the patient in need of treatment, and the severity of the nausea and/or vomiting experienced by the patient. An ordinary skilled physician can readily determine and prescribe the effective amount of a compound of formula II or prodrugs recited herein to prevent, improve or reduce pruritus and/or other diseases recited herein including nausea and/or emesis or chronic cough or OAB or alcohol dependency or depression. For example, the total daily dosage of the compound of formula II for an adult human is from 0.1 mg/kg to 4 mg/kg patient body weight or from 0.1 mg/kg to 3 mg/kg patient body weight (assuming a dosage range of 7-280 mg/day for a 70 kg patient). These doses may vary further based upon the particular disease being treated and the individual patient or group of patients.

The pharmaceutical composition may be orally administered or intravenously administered by infusion for a period of 15 to 90 minutes, 15 to 60 minutes, or 15 to 30 minutes. In some formulations, the composition may be administered by a bolus injection.

In another embodiment of the invention, an effective amount of one or more of the formulations recited herein may be used in combination with other active ingredients either in separate doses and before or after administration of the additional active ingredient (or concurrently therewith) or in fixed combination doses of the NK-1 antagonist (formula I or II or IIa-IIc) in combination with such other active ingredient. The formulations of the invention may be administered alone or in combination with one or more selective serotonin reuptake inhibitors (“SSRIs”) to treat depression or anxiety. Representative SSRIs include fluoxetine, fluvoxamine, paroxetine, sertraline and pharmaceutically acceptable salts thereof.

In another aspect, the invention relates to a method of treating emesis or delayed onset emesis such as that experienced hours to days after receiving chemotherapy. Combinations of the iv formulations of the present invention with another anti-emetic agent such as a serotonin 5-HT3 receptor antagonist, a corticosteroid or a substituted benzamide can be used to treat other forms of emesis including acute emesis induced by chemotherapy, radiation, motion and/or alcohol (ethanol), and post-operative nausea and vomiting. Examples of 5-HT3 antagonists include palosetron, dolasetron, ondansetron and granisetron or pharmaceutically acceptable salts thereof. An example of a suitable corticosteroid is dexamethasone. An example of a suitable benzamide is metoclopramide.

For cough, the compound of formula I, II or IIa-IIc may be combined with other treatments for cough. Such treatments include antitussives that are prescribed or over the counter cough medications. Preferred combinations include combinations of any two of the above or any one of the above with (or within) the NK-1 iv formulation or oral capsule formulation.

The present invention also relates to a method of treating acute and delayed nausea and vomiting associated with initial and repeat courses of moderately emetogenic cancer chemotherapy comprising administration of an intravenous formulation of a compound of formula I, II IIa, IIb or IIc in combination with at least one additional antiemetic agent.

In addition, each of the compounds according to formulas I, II IIa-IIc may be used for any NK-1 related disease or disorder or condition to treat a patient in need of treatment thereof. Such diseases and disorders include respiratory diseases, inflammatory diseases, skin disorders, ophthalmological disorders, central nervous system conditions, depression, anxiety, phobia, bipolar disorder, additions, alcohol dependence, psychoactive substance abuse, epilepsy, nociception, psychosis, schizophrenia, Alzheimer's disease, AIDS related dementia, Towne's disease, stress related disorders, obsessive/compulsive disorders, eating disorders, bulimia, anorexia nervosa, binge eating, sleep disorders, mania, premenstrual syndrome, gastrointestinal disorders, atherosclerosis, fibrosing disorders, obesity, Type II diabetes, pain related disorders, headache, neuropathic pain, post-operative pain, chronic pain syndrome, bladder disorders, genitourinary disorders, cough, emesis and nausea.

The present invention also relates to an oral formulation selected from a tablet, capsule or any suitable oral form. The preferred indication for the oral form includes, for example, cough or OAB (overactive bladder) or a neurological disease or disorder (e.g. depression). The Examples provided below describe preclinical and clinical data with respect to a compound of formula I in cough and OAB.

The compound of formula II is a potent, selective, competitive NK1 receptor antagonist [Ki]=0.28 nM and also competitively antagonizes functional effects mediated by activation of the NK1 receptor in cultured cells (equilibrium constant determined by Schild analysis [Kb]=0.17 nM. This compound is also a potent antagonist (effective dose for 90% inhibition [ED90]=0.2 mg/kg) in an in vivo pharmacodynamic model of NK1 receptor activity (gerbil foot thumping) and it also does not have significant affinity for NK2 or NK3 receptors (Ki>1 uM) or for a battery of other receptors, transporters, enzymes or ion channels.

The present invention comprises a formulation having a compound of formula II in a non-hygroscopic form. The compound of formula II (in free base form) is preferably a white to off-white powder and with a calculated pKa at 1.8; 7.0 and 7.6. The preferred form is a crystalline form of the free amine. The compound of formula II has a solubility at pH7 of 1.8 g/mL. The compound of formula II has an onset melting temperature at 168.9 as measured by differential scanning calorimetry (DSC). The compound of formula II was also prepared as an amorphous hydrochloride salt.

A preferred formulation comprises a compound of formula II and optional excipients including diluents, binders, disintegrants, wetting agents and lubricants. A preferred diluent is lactose monohydrate and/or microcrystalline cellulose; a preferred binder is povidone; a preferred disintegrant is crospovidone; a preferred wetting agent is poloxamer 188 and a preferred lubricant is magnesium stearate. Tablets or capsules may be prepared using 1-50 mgs of a compound of formula I with preferred strengths being 2.5 to 50 mgs and which may be administered once a day or multiple times per day with the maximum dose per day of about 200 mgs of active ingredient. This dosage and treatment regimen may vary depending upon the type of disease being treated and the individual patient. Dosage strengths in tablet or capsule form may be in 10, 25, 50, 100 or 200 mg strengths. The AUC plasma blood level from the 10 mg to 200 mg dose may range from about 19,000 to about 352,000 ng-hr/mL) after a single dose. Cmax values (either calculated or estimated) for the 10 mg to 200 mg dosage range were determined to be 103 to 1480 ng/mL and were based upon values obtained from healthy human volunteers administered 10, 25 and 50 mgs of SCH900978. Exposure multiples (EM) may be calculated to account for the total exposure produced following administration of 7 once-daily doses to humans in the following manner:

$$EM = \frac{AUC ( 0 \text{-} 24 hr ) , animals \times 29 days}{Total AUC over 7 doses , humans}$$

The EMs for the 10-50 mg dosage forms ranged from many multiples for the 10 mg dosage form to several multiples at the 50 mg dosage form (EM=19, 5 respectively; animal to human Cmax multiples).

In a preferred embodiment, the present invention is directed to the use of arylalkoxyalkylheteroaryl(aryl)piperidine carbonitriles in the treatment of pruritus or prurigo nodularis, preferably the use of a compound having formula II:

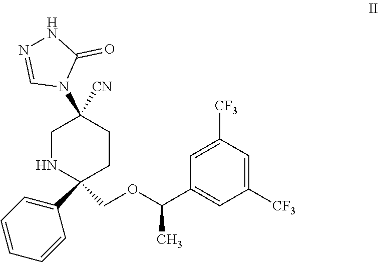 [SMILES: C[C@@H](OC[C@@]1(c2ccccc2)CC[C@](C#N)(n2cn[nH]c2=O)CN1)c1cc(C(F)(F)F)cc(C(F)(F)F)c1]

(3 S,6S)-6-({(1R)-1-[3,5-bis(trifluoromethyl)phenyl]ethoxy}methyl)-3-(5-oxo-1,5dihydro-4H-1,2,4-triazol-4-yl)-6-phenylpiperidine-3-carbonitrile and pharmaceutically acceptable salts and/or solvates thereof. This compound may be produced as shown for the preparation of compound 42 in U.S. Pat. No. 7,709,641. In addition, this compound in crystalline form may be produced by the following method:
The compound of formula II in crystalline form may be prepared according to the following Scheme 1:

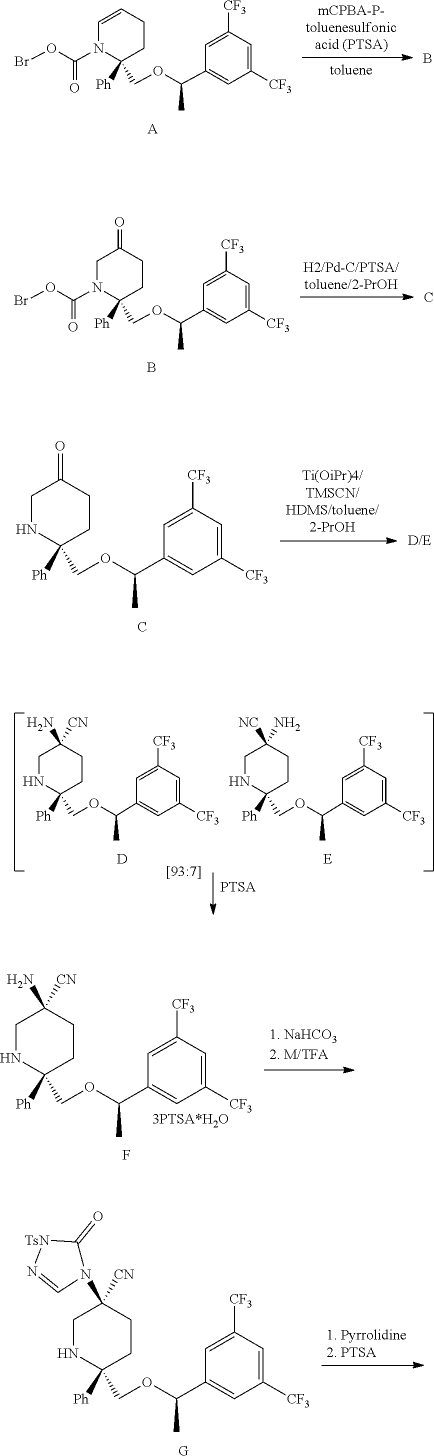

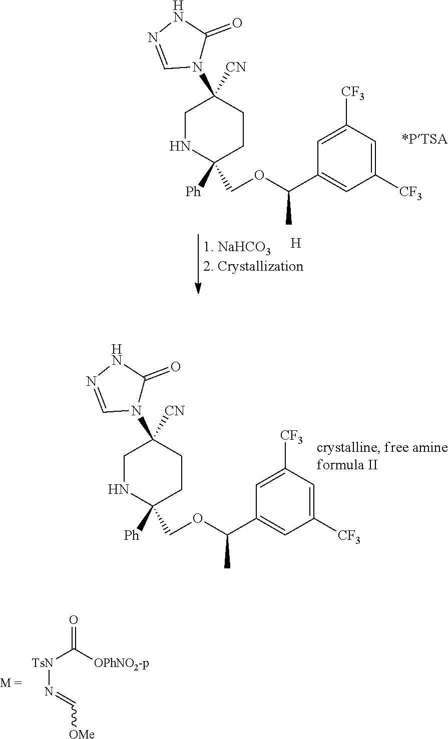

Intermediate A was reacted in toluene with no more than 1 equivalent of mCPBA at below 30 degrees C. to form the epoxide, which was then treated with a catalytic amount of PTSA at 75 degrees C. to give the alpha-amino ketone B. Upon complete conversion the batch was worked up by the addition of water and the pH was adjusted to 13 with sodium hydroxide to remove acid by-products. The organic phase was washed with brine to reach pH 7-8 in order to run the subsequent hydrogenation. Intermediate B was hydrogenated using hydrogen/Palladium on carbon with 1.5 equivalents of MeSO₃H (methanesulfonic acid) in toluene/isopropanol to remove the Cbz protecting group to form Intermediate C. The reaction was run for 4-5 hours at 20-25 degrees C. under 1.5 Bar of hydrogen pressure. The batch was cooled after complete conversion to 0-10 degrees C. The catalyst was removed by filtration and the filtrate was used directly in the subsequent reaction. A Strecker synthesis was then performed to form the key alpha amino nitrile intermediate D. HMDS (hexamethyldisilazane or [(CH₃)₃Si]₂NH); T1(OiPr)₄; TMSCN; toluene/2-PrOH; HCl and NaOH are used with intermediate C to form intermediate D (not isolated)/F (isolated at the 3pTsOH*H2O salt) in high yield and with high diastereoselectivity. The biselectrophilic reagent M was then used to react with F to form the key triazolinone compound G which was detosylated with pyrrolidine and treated with PTSA to form the penultimate intermediate H—the tosylate salt of a compound of formula I which was then treated with NaHCO₃ and crystallized to form the crystalline free amine form of a compound of formula II. The present invention relates to novel salt forms including intermediate D in the form of the 3pTsOH*H20 hydrate and the pTsOH salt of a compound of formula I. The present invention also relates to a process for making a crystalline compound of formula I comprising treating a pTsOH salt of formula I with mild base (NaHCO₃). The present invention also relates to a process for making a compound of formula I comprising using intermediate A to form B; B to form C; C to form D; D to form F: F to form G; and G to form H and H to form a compound of formula II. The starting compound A may be prepared as described in U.S. Pat. No. 7,049,320 as described therein for the synthesis of compound A59 therein from compound A58 and so on.

In general, the efficacy of the compounds of the invention can be evaluated in both animal models of pruritus and in human clinical studies. These studies are conducted under laws, rules and regulations set forth in the United States Code and under the supervision of the U.S. Food and Drug Administration. The general safety of a drug is determined in phase I clinical trials while phase II trials assess safety and efficacy. Phase III trials are larger trials used to gather additional efficacy information and information regarding the safety of the drug. Patients having pruritus have their own perceptions about the “cure” and treatment of the condition. The tools and tests used in clinical trials to evaluate the effectiveness of a drug include the Visual Analog Scale (VAS)—see Phan et al. Acta Derm. Venereal., 2012; 92:502-507). This test is a graphic measurement with a 100 mm horizontal line labeled on the left side as “no symptom” and on the right side as “worst imaginable symptom.” Patients are asked to draw a vertical line on a point on the horizontal line to indicate the degree or intensity of the symptom. The length from the left end to the point marked by the patient is measured in millimeters. The results are then analyzed using standard statistical methods and according to clinical trial protocol. Other accepted methods to assess the effectiveness of a drug to treat pruritus include the Dermatology Life Quality Index (DLQI). This test is a self-administered questionnaire developed and published at the University of Hospital of Wales. See Finlay et al, Clin. Exper. Derm., 1994, 19:210-216. See also Hahn et al., J. Am. Acad. Dermatol., 2001, 45(1):44-48.

### EXAMPLES

### Example 1 Preparation of (3 S,6S)-6-((1R)-1-[3,5-bis(trifluoromethyl)phenyl]ethoxy}methyl)-3-(5-oxo-1,5dihydro-4H-1,2,4-triazol-4-yl)-6-phenylpiperidine-3-carbonitrile

The process disclosed in U.S. Pat. No. 7,709,641 for the preparation of compound 42 therein may be followed to produce the compound of formula II. The ketone intermediate (compound 41a therein) (6.87 g, 11.86 mmol) in ethanol (7 mL) is added to a solution of NaCN (0.767 g), NH₄Cl (0.889 g) and NH₃H₂O (3.84 mL) in EtOH (7.0 mL) and water (7.0 mL) at room temperature in a sealed tube. The sealed tube is then heated at 60° C. for 12 hours before it is cooled down to room temperature. The reaction mixture is diluted with EtOAc (200 mL) and washed with water (50 mL). The aqueous phase is extracted with EtOAc (3×30 mL). The combined organic layers are washed with brine (30 mL), and dried over MgSO₄. After filtration and concentration, the crude product is purified using BIOTAGE chromatography (hexane/EtOAc, v/v=7/2 to 5/2) to give the compounds shown as 42b and 42c in U.S. Pat. No. 7,709,641. The compound 42b is then carried forward in step B to form Compound 42d. Phosgene (6.67 mL, 12.4 mmol, 20% in toluene) is added dropwise to a vigorously stirred mixture of Compound 42b (1.5 g, 2.48 mmol) in CH₂Cl₂ (30 mL) and a saturated NaHCO₃ solution (30 mL) at 0° C. The mixture is stirred at 0° C. for 3 hours and then diluted with CH₂Cl₂ (50 mL) and the aqueous phase is separate from the organic phase. The organic phase is washed with a cold aqueous NH₄Cl solution, brine, and dried over MgSO₄. The solvent is reduced to a volume of about 5 mL under reduced pressure, at room temperature, to remove excess phosgene. The residue is dissolved in CH₂Cl₂ (15 mL) and treated with NH₂NHC(O)H (0.446 g, 7.44 mmol) and pyridine (1.2 mL, 14.88 mmol) at room temperature.

The resulting solution is stirred at room temperature for 12 hours. The reaction mixture is then diluted with EtOAc (200 mL) and washed with HCl (50 mL, 0.5 N). The aqueous phase is extracted with EtOAc (3×30 mL). The combined organic layers are washed with brine (30 mL), and dried over MgSO₄. After filtration and concentration, the crude product is purified using BIOTAGE chromatography eluted with hexane/EtOAc (v/v=1/2 to 1/7) to give Compound 42d as shown in U.S. Pat. No. 7,709,641. This intermediate is then used to prepare Compound 42 shown in the '641 patent or a compound of formula II above. TMSCl (trimethylsilylchloride) (50 uL) is added to a stirring mixture of Compound 42d (15 mg, 0.022 mmol) and LiI (lithium iodide) (2.9 mg, 0.028 mmol) in HDMS (0.5 mL) at room temperature. The resulting reaction mixture is heated to about 140° C. for 30 hours before it is cooled down to room temperature. The reaction mixture is diluted with EtOAc (25 mL) and washed with HCl (5 mL, 1.0 N). The aqueous phase is extracted with EtOAc (3×30 mL). The combined organic extracts are washed with brine (10 mL), and dried over MgSO₄. After filtration and concentration, the crude product is purified using preparative TLC (hexane/EtOAc, v/v 6/4) to give compound 42 (or formula II herein). Alternative methods may also be used to synthesize a compound of formula II including those methods described in the 7,709,641 patent using, for example, the intermediate described therein as Compound 23g (R²═CH₂OH) which is oxidized to form Compound 42e (R²═—C(O)H) which is further reacted with hydroxylamine hydrochloride to form compound 42g (R²═C═N—OH) which is then treated with 1,1′oxyalydimidazole in benzene to form compound 42 (R²═—CN). This compound is also known as SCH900978. In addition, salts may be prepared as well.

### Example 2

A series of in vitro and in vivo studies examined the effects of SCH900978 on cardiovascular, respiratory and CNS functions. No adverse effects were observed. The NOAEL was ≥25 mg/kg in male rats, ≥12.5 mg/kg in female rats and ≥10 mg/kg in male cynomolgus monkeys; each dose was the highest dose tested. SCH900978 (compound of formula I) produced a concentration-dependent inhibition of the hERG gene current with a measured IC50 of 860 nM. These data suggested that a delay in cardiac repolarization would not be observed at projected clinical doses.

### Example 3

Following administration of a single oral dose of SCH900978 to rats and monkeys, the plasma concentration-time profiles showed extended absorption of the drug and a long apparent terminal elimination phase half-life (t½). Exposure to SCH900978 after once daily oral doses for one month in rats and monkeys generally increased as the dose increased. Exposure to SCH900978 was higher in female rats than in male rats, but was independent of sex in monkeys. At low doses in the toxicology/toxicokinetic studies, plasma SCH 900978 concentrations were greater over the 24-hour period following administration of the 29^{th} dose than following administration of the first dose in female rats (0.5 to 12.5 mg/kg), male rats (1 to 5 mg/kg) and monkeys (0.5 to 30 mg/kg). At doses ≥25 mg/kg in female and male rats, systemic exposure to SCH 900978 decreased following repeated administration, most likely due to cytochrome P450 (CYP) enzyme induction.

### Example 4

Protein binding studies were conducted and found that a compound of formula I was highly protein bound (≥99.6%) in all mammals tested as well as human plasma and readily crossed the blood brain barrier (rats, gerbils and guinea pigs). The major metabolic pathways in the mammals tested involved oxidation and glucuronidation of a compound of formula II. After oral administration in rats and monkeys the major elimination pathway was excretion into the feces. Urinary excretion was also substantial (approximately 20%).

### Example 5

A randomized, placebo-controlled, third party blind (within dose level), rising, single-dose escalation study to evaluate the safety, tolerability and pharmacokinetics of SCH900978 in healthy human volunteers was conducted and completed. Three dose cohorts of six healthy volunteers each received single oral doses of 10, 25, or 50 mgs of SCH900978. Six subjects received placebo. The study found that single doses of up to 50 mgs of a compound of formula II were safe and well tolerated. Pharmacokinetic analyses showed that SCH900978 was rapidly absorbed and slowly eliminated. The t½ was approximately 10 to 13 days. Exposure to SCH 900978 (expressed by Cmax and AUC) was dose related and variability was low (% coefficient of variation [CV] was 8-32%). The bioavailability of SCH900978 was comparable between solution and capsule oral formulations. Food had no effect on the oral bioavailability of SCH900978. Mean plasma concentrations of SCH900978 following administration of a single oral dose of 10, 25 and 50 mgs of the compound 0-24 hours post dose ranged between >0-100 to about 400 ng/mL. Tmax occurred within 0-4 hours at each dosage strength. Table 2 below provides the mean SCH900978 pharmacokinetic parameters following administration of a single oral dose of 25 mgs of SCH900978 capsule or solution to healthy volunteers.

TABLE 2

| | | | AUC(tf) | AUC(l) | | |
|---|---|---|---|---|---|---|
| | Cmax (ng/mL) | Tmax (hr) | (ng-hr/mL) | (ng-hr/mL) | T½ (hr) | Tf (hr) |
| Treatment | Mean/% CV | Med/range | Mean/% CV | Mean/% CV | Mean/% CV | Mean/% CV |
| 25 mg | 213/27 | 2.5/1.5-4 | 25700^{b}/17 | 42900^{c}/13 | 238^{c}/26 | 312^{b}/0 |
| capsule (n = 6) | | | | | | |
| 25 mg | 301/21 | 1.5/1-2 | 29900/22 | 52600^{c}/25 | 253^{c}/15 | 312/0 |
| solution^{a}(n = 5) | | | | | | |

Cmax = maximum observed plasma concentration;
Tmax = time to maximum observed plasma concentration;
AUC(tf) = area under the plasma concentration-time curve from time 0 to time of final quantifiable sample;
AUC(l) = area under the plasma concentration-time curve from time 0 to infinity;
t½ = apparent terminal elimination phase half-life;
tf = time of final quantifiable sample;
CV = coefficient of variation.
^{a}Individual plasma concentrations were adjusted for carryover effect by subtracting extrapolated plasma SCH900978 concentration from Period 1 (capsule formulation) at the corresponding time points.
^{b}n = 5; tf for one subject was 120 hours. AUC(tf) for this subject was not included in calculation of the mean.
^{c}n = 4; terminal elimination phase could not be determined for two subjects because t½ was >tf.

Table 3 below provides the mean SCH900978 pharmacokinetic parameters following administration of a single oral dose of 50 mgs of SCH900978 to healthy volunteers under fasted and fed conditions.

TABLE 3

| | Cmax | | AUC(tf) | AUC(l) | | |
|---|---|---|---|---|---|---|
| | (ng/mL) | Tmax(hr) | (ng-hr/mL) | (ng-hr/mL) | T½ (hr) | Tf (hr) |
| Treatment | Mean/% CV | Median/range | Mean/% CV | Mean/% CV | Mean/% CV | Mean/% CV |
| 50 mg | 388/32 | 3/3-4 | 59700/24 | 88700/23 | 309/32 | 504/0 |
| fasted | | | | | | |
| (n = 6) | | | | | | |
| 50 mg | 366/23 | 6/4-10 | 69000/19 | 97000^{b}/16 | 316^{b}/38 | 504/0 |
| fed^{a} (n = 6) | | | | | | |

^{a}individual plasma concentration were adjusted for carryover effect by subtracting extrapolated plasma sCH900978 concentrations from Period 1 (fasted state) at the corresponding time points.
^{b}n = 4; termination elimination phase could not be determined for two subject because t½ was >tf.

### Example 6

Radioligand binding studies were conducted with SCH900978 and it was found to bind with high affinity to NK1 receptors in humans and other mammalian species as demonstrated by its ability to block NK1 receptor agonist (GR-73632)-mediated stimulation of calcium efflux in cells expressing the human NK1 receptor (Kb=0.17 nM). Human (Ki=0.28); Gerbil (0.2); Rat (4.62) and Mouse (0.76). SCH900978 was also tested for NK2 and NK3 receptor activity and in 106 other receptors, transporters, enzymes and ion channels and had greater than 1000 fold lower affinity for these than for the NK1 receptor.

### Example 7

Intracerebroventricular administration of NK1 receptor agonists to gerbils induces vigorous thumping in the hind feet. This response can be blocked by NK1 receptor antagonists. When administered orally six hours before testing, SCH900978 blocked NK1 receptor agonist (GR 73632-induced foot thumping in a dose-dependent manner (ED90=0.2 mg/kg). In a subsequent study, SCH900978 (0.3 mg/kg oral dose) significantly inhibited NK1 receptor agonist-induced foot thumping for up to 48 hours.

### Example 8

SCH900978 was tested in two animal models of cough: irritant (capsaicin)-induced cough in guinea pig and mechanically induced cough in dog. Inhalation of the irritant capsaicin induces coughing in both animals and humans, in part by releasing substance P from unmyelinated sensory C fibers in the lung. When administered orally to conscious guinea pigs six hours prior to inhalation of aerosolized capsaicin, SCH900978 attenuated capsaicin-induced cough in a dose-dependent manner (ED90=0.04 mg/kg) (FIG. 2). The maximal inhibit of capsaicin-induced cough produced by SCH900978 was comparable to that produced by the standard antitussive agents codeine, dextromethorphan, hydrocodone and baclofen (55% to 60% inhibition).

The antitussive activity of SCH 900978 was also evaluated against cough induced by mechanical stimulation of the intrathoracic trachea in anesthetized dogs. Cough was measured in each stimulation trial by the cough frequency (number of coughs) and cough amplitude (cm H2O increase in expiratory pressure). Oral administration of SCH900978 (0.3 mg/kg) significantly reduced both cough frequency and amplitude for up to 24 hours (FIG. 3). The 0.3 mg/kg oral dose of SCH 900978 maximally inhibited the reduction in blood pressure and the increase in minute volume elicited by the NK1 receptor agonist substance P in dogs; consequently, this dose can be considered to be a maximally effective dose. Plasma concentration of SCH900978 at 2, 6 and 25 hours after administration of the 0.3 mg/kg dose to dogs were 201, 151 and 102 ng/mL (373, 280 and 189 nM), respectively.

### Example 9

Following single-dose administration of SCH 900978, the plasma concentration-time profiles in rats and monkeys showed extended absorption of SCH900978 with a long t1/2. The t1/2 in male cynomolgus monkeys ranged from 16 to 33 hours after oral administration of 0.3 or 1.0 mg/kg of SCH 900978 amorphous hydrochloride. Systemic exposure area under the plasma concentration-time curve [AUC] to SCH900978 following a single dose increased with increasing dose from 0.6 to 500 mg/kg in male rats and from 0.5 to 250 mg/kg in female rats but did not increase further at the highest doses. In monkeys, systemic exposure to SCH900978 following a single dose increased with increasing dose.

### Example 10

The extent of brain penetration by SCH 900978 was evaluated after oral administration of SCH 900978 amorphous hydrochloride to female gerbils (0.1 mg/kg), male Sprague Dawley rats (5 mg/kg), and male guinea pigs (0.3 mg/kg). SCH900978 was observed in the brains of all three species; brain concentrations were similar to or greater than plasma concentrations.

### Example 11—Synthesis of Crystalline, Free Form of a Compound of Formula II

Intermediate A as shown in Scheme 1 was reacted in toluene with no more than 1 equivalent of mCPBA at below 30 degrees C. to form the epoxide, which was then treated with a catalytic amount of PTSA at 75 degrees C. to give the alpha-amino ketone B. Upon complete conversion the batch was worked up by the addition of water and the pH was adjusted to 13 with sodium hydroxide to remove acid by-products. The organic phase was washed with brine to reach pH 7-8 in order to run the subsequent hydrogenation. Intermediate B was hydrogenated using hydrogen/Palladium on carbon with 1.5 equivalents of MeSO₃H (methanesulfonic acid) in toluene/isopropanol to remove the Cbz protecting group to form Intermediate C. The reaction was run for 4-5 hours at 20-25 degrees C. under 1.5 Bar of hydrogen pressure. The batch was cooled after complete conversion to 0-10 degrees C. The catalyst was removed by filtration and the filtrate was used directly in the subsequent reaction. A Strecker synthesis was then performed to form the key alpha amino nitrile intermediate D. HMDS (hexamethyldisilazane or [(CH₃)₃Si]₂NH); Ti(OiPr)₄; TMSCN; toluene/2-PrOH; HCl and NaOH are used with intermediate C to form intermediate D (not isolated)/F (isolated at the 3pTsOH*H2O salt) in high yield and with high diastereoselectivity. The biselectrophilic reagent M was then used to react with F to form the key triazolinone compound G which was detosylated with pyrrolidine and treated with PTSA to form the penultimate intermediate H—the tosylate salt of a compound of formula I which was then treated with NaHCO₃ and crystallized to form the crystalline free amine form of a compound of formula II. The present invention relates to novel salt forms including intermediate D in the form of the 3pTsOH*H20 hydrate and the pTsOH salt of a compound of formula II. The present invention also relates to a process for making a crystalline compound of formula II comprising treating a pTsOH salt of formula II with mild base (NaHCO₃). The present invention also relates to a process for making a compound of formula II comprising using intermediate A to form B; B to form C; C to form D; D to form F: F to form G; and G to form H and H to form a compound of formula II.

### Example 12 Animal Models of Pruritus

The compounds of the invention are administered in suitable dosages in animal models of pruritus which include several mouse models. A compound of the invention, including, for example, a compound of formula II, is provided to a group of mice in which 4-ethoxymethylene-2-phenyl-2-oxazolin-5-one has been provided to induce chronic dermatitis with an associated itch response. See J. Pharmacol. Sci. 2010, 113:255-262). Alternatively or, in addition, other animal models which use spider venom (Costa et al, Vascul. Pharmacol., 2006, 45(4):209-14 or picrylchloride (Ohmura et al. Eur. J. Pharmacol., 2004; 491:191-194) are used to evaluate the antipruritic activity of the compounds according to the invention. In each case, after itching is induced in the test subject, the test compound and a placebo is administered to the subject animal to analyze for scratching behavior using standard statistical methods. The compounds are considered effective if continuous itching and/or scratching is militated.

### Example 13 Pre-Clinical and Clinical Studies of a Compound of Formula II and Pruritus

The effectiveness of a compound of formula II and other compounds described herein to treat pruritus can be shown in well controlled clinical trials. Multiple pre-clinical and clinical safety studies have been conducted with the compound of formula II (SCH900978) as recited above. A human clinical trial testing the efficacy of different doses of a compound of formula II is conducted under FDA requirements. The study is a phase II study and is randomized, double blinded, placebo controlled. The patients include adult males and females in ages of between 18 and 72 years old. The patients are previously diagnosed with chronic pruritus and which are unresponsive to standard treatment. Chronic pruritus is generally defined as having itching of a six week period or longer and a VAS score of greater than 7. The patients are randomized to receive one of several doses of active ingredient (a compound of formula II or salt thereof) or placebo. The study will be conducted over a two to eight week period and patients will receive the drug once per day. An initial loading dose of 3× the subsequent doses may be given on the first day of the study. The doses tested are 10 mg, 25 mg and 50 mgs of SCH900978 once per day. Clinical studies in humans showed these doses were safe and well tolerated. The number of subjects enrolled in the study ranges from 80 to 240. The primary endpoint of the study is measured by a change in VAS score between the drug doses and the placebo. Secondary endpoints are measured using the DLQI index, lesion healing and patient and physician assessments. Safety evaluation is also being conducted.

It will be appreciated that other studies including those for prurigo nodularis are conducted in well controlled clinical trials.

What is claimed:

1. A method of treating pruritus or prurigo nodularis in a patient in need of treatment thereof comprising administering a pharmaceutically effective amount of a compound of formula I or a pharmaceutically acceptable salt or solvate thereof, the compound of formula I having the formula: 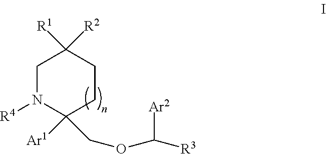
wherein:
R1 and R2 are independently selected from the group consisting of H, alkyl, haloalkyl, alkyl substituted with one or more hydroxyl groups, —CN, alkynyl, —N(R⁶)₂, —N(R⁶)—S(O₂)alkyl, —N(R⁶)—C(O)—N(R⁹)₂, -alkylene-CN, cycloalkylene-CN, -alkylene-O-alkyl, —C(O)-alkyl, —C(═N—OR⁵)-alkyl, —C(O)—N(R⁹)₂, —C(O)—O-alkyl, -alkylene-C(O)-alkyl, -alkylene-C(O)—O-alkyl, -alkylene-C(O)—N(R⁹)₂,
With the proviso that at least one of R¹ and R² is —CN, 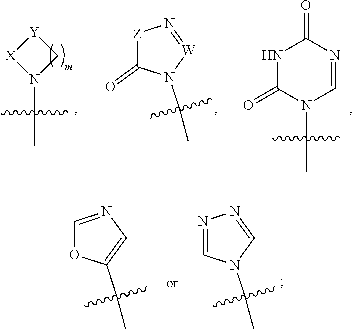
W is ═C(R⁸)— or ═N—;
X is —C(O)— or —S(O₂)—;
Y is selected from the group consisting of —CH₂—, —O—, and —N(R⁶)—C(O)—, with the proviso that:
(a) The nitrogen atom of —N(R⁶)—C(O)— is bonded to X, and
(b) If R¹ and/or R² is 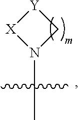
And Y is —O—, X is not —S(O₂)—;
Z is —C(R⁷)₂—, —N(R⁶)—, or —O—;
R³ is selected from the group consisting of H, —CH₂OR⁵, and alkyl;
R⁴ is selected from the group consisting of H, alkyl, cycloalkyl, heterocycloalkyl, heteroaryl, aryl, acyl, aroyl, alkylsulfonyl, and arylsulfonyl;
R⁵ is H or alkyl;
R⁶ is selected from the group consisting of H, alkyl, cycloalkyl, and aryl;
Each R⁷ is independently H or alkyl; or
Each R⁷, together with the ring carbon to which they are shown attached, form a cycloalkylene ring;
R⁸ is selected from the group consisting of H, alkyl, alkyl substituted with one or more hydroxyl groups, —N(R⁶)₂, —N(R⁶)—S(O₂)-alkyl, —N(R⁶)—S(O₂)-aryl, —N(R⁶)—C(O)-alkyl, —N(R⁶)—C(O)-aryl, alkylene-O-alkyl, and —CN;
R⁹ is selected from the group consisting of H, alkyl, and aryl, or each R⁹, together with the nitrogen to which they are shown attached, form a heterocycloalkyl ring;
Ar¹ and Ar² are each independently selected from the group consisting of unsubstituted aryl and aryl substituted with 0 to 3 substituents selected from the group consisting of halogen, alkyl, alkoxy, haloalkyl, haloalkoxy, —CN, —OH, and —NO₂;
n is 0, 1, or 2; and
m is 1, 2 or 3.

2. The method according to claim 1 wherein in a compound of formula I, wherein
R³ is C₁₋₆ alkyl;
R⁴ is H;
Ar¹ is phenyl;
Ar² is phenyl substituted with 1 to 3 substituents selected from the group consisting of halogen, C₁₋₆ alkyl, C₁₋₆ alkoxy, C₁₋₆ haloalkyl, C₁₋₆ haloalkoxy, —CN, and —NO₂; and n is 1.

3. A method of treating pruritus in a patient in need of treatment thereof comprising administering a pharmaceutically effective amount of a compound of formula IA: 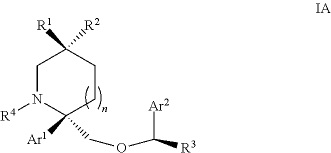
wherein,
R¹ and R² are independently selected from the group consisting of H, alkyl, haloalkyl, alkyl substituted with one or more hydroxyl groups, —CN, alkynyl, —N(R⁶)₂, —N(R⁶)—S(O₂)alkyl, —N(R⁶)—C(O)—N(R⁹)₂, -alkylene-CN, cycloalkylene-CN, -alkylene-O-alkyl, —C(O)-alkyl, —C(═N—OR⁵)-alkyl, —C(O)—N(R⁹)₂, —C(O)—O-alkyl, -alkylene-C(O)-alkyl, -alkylene-C(O)—O— alkyl, -alkylene-C(O)—N(R⁹)₂,
with the proviso that at least one of R¹ and R² is —CN, 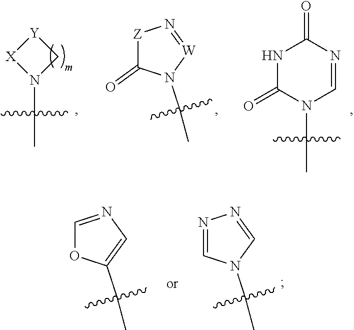
W is ═C(R⁸)— or ═N—;
X is —C(O)— or —S(O₂)—;
Y is selected from the group consisting of —CH₂—, —O—, and —N(R⁶)—C(O)—, with the proviso that:
(a) The nitrogen atom of —N(R⁶)—C(O)— is bonded to X, and
(b) If R¹ and/or R² is 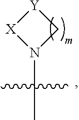
And Y is —O—, X is not —S(O₂)—;
Z is —C(R⁷)₂—, —N(R⁶)—, or —O—;
R³ is C₁₋₆ alkyl;
R is H;
Ar¹ is phenyl;
Ar² is phenyl substituted with 1 to 3 substituents selected from the group consisting of halogen, C₁₋₆ alkyl, C₁₋₆ alkoxy, C₁₋₆ haloalkyl, C₁₋₆ haloalkoxy, —CN, and —NO₂; and n is 1 and pharmaceutically acceptable salts and/or solvates thereof.

4. The method according to claim 3 wherein:
R¹ and R² are each independently selected from the group consisting of H, —CH₃, —CH₂—CH₂—CH₃, —CH₂C₁, —CH₂F, —CHCl₂, —CHF₂, —CF₃, —CH₂OH, —CH₂CH₂OH, —CH₂CH(OH)CH₃, —CH₂C(OH)(CH₃)₂, —CN, —CH₂CN, —NH₂, —NH—S(O₂)—CH₃, —NH—C(O)—NH₂, —CH₂OCH₃, —C(O)CH₃, —C(O)CH₂CH₃, —C(═N—OH)—CH₃, —C(═N—OH)—CH₂CH₃, —C(═N—OCH₃)—CH₃, —C(O)—NH₂, —C(O)NH(CH₃), —C(O)—O—CH₃, —CH₂—C(O)O—CH₃, —CH₂—C(O)OCH₂CH₃, —CH₂C(O)—NH(CH₂CH₃, —CH₂C(O)—NH₂, 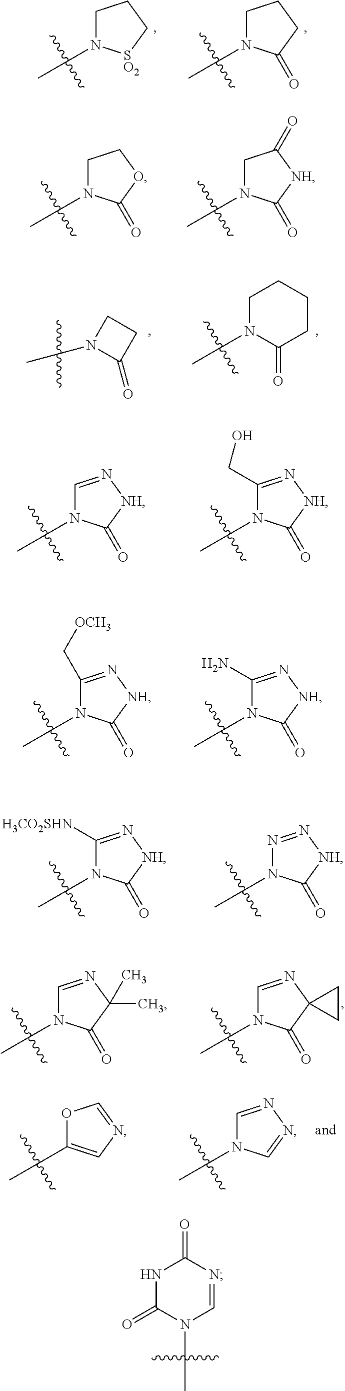
R³ is —CH₃;
R⁴ is H;
Ar¹ is phenyl;
Ar² is phenyl substituted with 1 to 3 substituents selected from the group consisting of halogen, C₁₋₆ alkyl, C₁₋₆ haloalkyl, C₁₋₆ haloalkoxy, —CN, and —NO₂; and n is 1 and pharmaceutically acceptable salts and/or solvates thereof.

5. A method of treating pruritus in a patient in need of treatment thereof comprising administering a pharmaceutically effective amount of a compound of formula IB wherein each of R¹ and R² are selected from:
IB

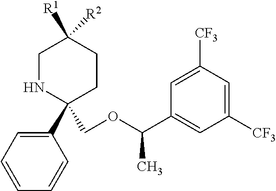

| Com- | | |
|---|---|---|
| pound | R^{l} | R² |
| 1 | 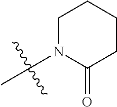 [SMILES: *N1CCCCC1=O] | —CN |
| 2 | 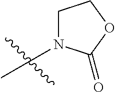 [SMILES: *N1CCOC1=O] | —CN |
| 3 | 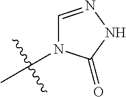 [SMILES: *n1cn[nH]c1=O] | 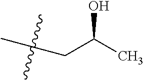 [SMILES: *C[C@H](C)O] |
| 4 | 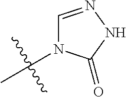 [SMILES: *n1cn[nH]c1=O] | 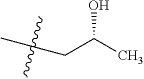 [SMILES: *C[C@@H](C)O] |
| 5 | 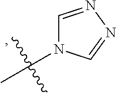 [SMILES: *n1cnnc1] | —CH₂CN |
| 6 | —CH₃ | 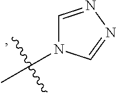 [SMILES: *n1cnnc1] |
| 7 | —CN | 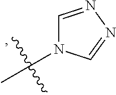 [SMILES: *n1cnnc1] |
| 8 | —C(O)OCH₃ | 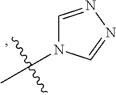 [SMILES: *n1cnnc1] |
| 9 | 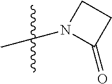 [SMILES: *N1CCC1=O] | —CN |
| 10 | —C(O)NH₂ | 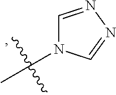 [SMILES: *n1cnnc1] |
| 11 | 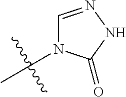 [SMILES: *n1cn[nH]c1=O] | —CH₂C(OH)(CH₃)₂ |
| 12 | —CH₂OH | 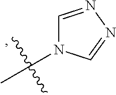 [SMILES: *n1cnnc1] |
| 13 | 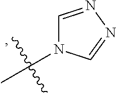 [SMILES: *n1cnnc1] | —CH₂OCH₃ |
| 14 | —CH₂OCH₃ | 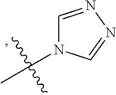 [SMILES: *n1cnnc1] |
| 15 | 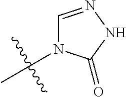 [SMILES: *n1cn[nH]c1=O] | —CH₂NH—S(O₂)CH₃ |
| 16 | 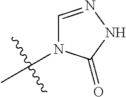 [SMILES: *n1cn[nH]c1=O] | —CH₂C(O)NH(CH₂CH₃) |
| 17 | 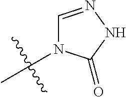 [SMILES: *n1cn[nH]c1=O] | —CH₂C(O)OCH₂CH₃ |
| 18 | 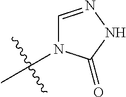 [SMILES: *n1cn[nH]c1=O] | —C(═N—OH)—CH₂CH₃ |
| 19 | 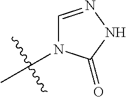 [SMILES: *n1cn[nH]c1=O] | —C(O)CH₂CH₃ |
| 20 | 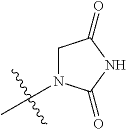 [SMILES: *N1CC(=O)NC1=O] | —CH₂OCH₃ |
| 21 | 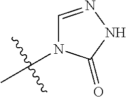 [SMILES: *n1cn[nH]c1=O] | —C(O)NH(CH₃) |
| 22 | —C(O)NH(CH₃) | 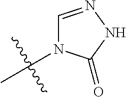 [SMILES: *n1cn[nH]c1=O] |
| 23 | 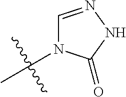 [SMILES: *n1cn[nH]c1=O] | —CH₂OH |
| 24 | 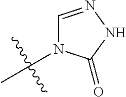 [SMILES: *n1cn[nH]c1=O] | —CH₂CH₂OH |
| 25 | 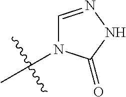 [SMILES: *n1cn[nH]c1=O] | —CH₂CH₂CH₃ |
| 26 | 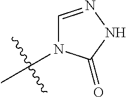 [SMILES: *n1cn[nH]c1=O] | —CH₂OCH₃ |
| 27 | —CH₂OCH₃ | 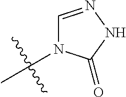 [SMILES: *n1cn[nH]c1=O] |
| 28 | 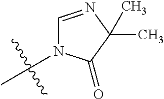 [SMILES: *N1C=NC(C)(C)C1=O] | H |
| 29 | 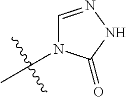 [SMILES: *n1cn[nH]c1=O] | —CH₂C(O)NH₂ |
| 30 | 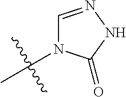 [SMILES: *n1cn[nH]c1=O] | —CH₂C(O)CH₃ |
| 31 | 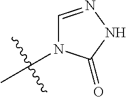 [SMILES: *n1cn[nH]c1=O] | —CH₂C(O)OCH₃ |
| 32 | 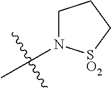 [SMILES: *N1CCCS1(=O)=O] | —CN |
| 33 | 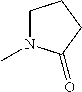 | —CN |
| 34 | —CN | 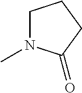 |
| 35 | —CN | 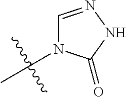 [SMILES: *n1cn[nH]c1=O] |
| 36 | —NHS (O₂)CH₃ | —CN |
| 37 | —CN | —NHS(O₂)CH₃ |
| 38 | 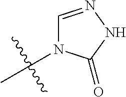 [SMILES: *n1cn[nH]c1=O] | —CH₂CN |
| 39 | —CN | —NH₂ |
| 40 | —NH₂ | —CN |
| 41 | —NHC(O)NH₂ | —CN |
| 42 | 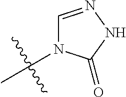 [SMILES: *n1cn[nH]c1=O] | —CN |
| 43 | 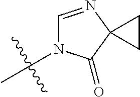 [SMILES: *N1C=NC2(CC2)C1=O] | H |
| 44 | 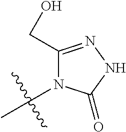 [SMILES: *n1c(CO)n[nH]c1=O] | H |
| 45 | 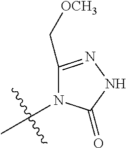 [SMILES: *n1c(COC)n[nH]c1=O] | H |
| 46 | H | 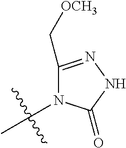 [SMILES: *n1c(COC)n[nH]c1=O] |
| 47 | 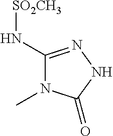 | H |
| 48 | H | 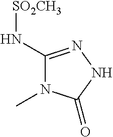 |
| 49 | 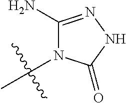 [SMILES: *n1c(N)n[nH]c1=O] | H |
| 50 | H | 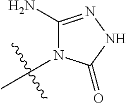 [SMILES: *n1c(N)n[nH]c1=O] |
| 51 | 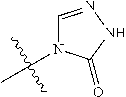 [SMILES: *n1cn[nH]c1=O] | —C(O)NH₂ |
| 52 | 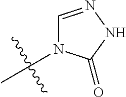 [SMILES: *n1cn[nH]c1=O] | —C(═N—OCH₃)—CH₃ |
| 53 | 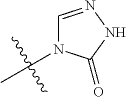 [SMILES: *n1cn[nH]c1=O] | —C(O)CH₃ |
| 54 | 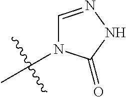 [SMILES: *n1cn[nH]c1=O] | —C(═N—OH)—CH₃ |
| 55 | 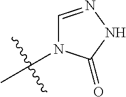 [SMILES: *n1cn[nH]c1=O] | —C(O)OCH₃ |
| 56 | 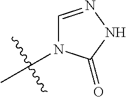 [SMILES: *n1cn[nH]c1=O] | —CH₂Cl |
| 57 | 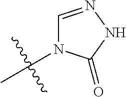 [SMILES: *n1cn[nH]c1=O] | —CH₃ |
| 58 | 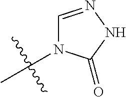 [SMILES: *n1cn[nH]c1=O] | —C(═N—OCH₃)—CH₂CH₃ |
| 59 | —NHC(O)CH₃ | 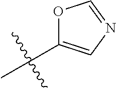 [SMILES: *c1cnco1] |
| 60 | H | 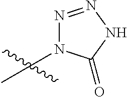 [SMILES: *n1nn[nH]c1=O] |
| 61 | 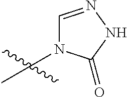 [SMILES: *n1cn[nH]c1=O] | H |

6. A method of treating chronic pruritus in a patient in need of treatment thereof comprising administering a pharmaceutically effective amount of a compound of formula II 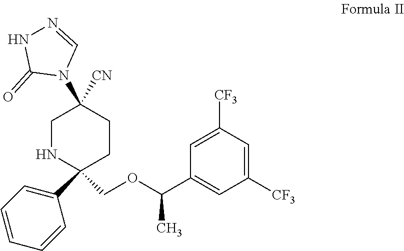 [SMILES: C[C@@H](OC[C@@]1(c2ccccc2)CC[C@](C#N)(n2cn[nH]c2=O)CN1)c1cc(C(F)(F)F)cc(C(F)(F)F)c1]
or a pharmaceutically acceptable salt thereof.

7. The method according to claim 6 wherein the compound of formula II is in the form of a crystalline free amine.

8. The method according to claim 6 wherein the compound of formula II is an amorphous hydrochloride salt.

9. A pharmaceutical composition comprising a pharmaceutically effective amount of a crystalline form of compound of formula II 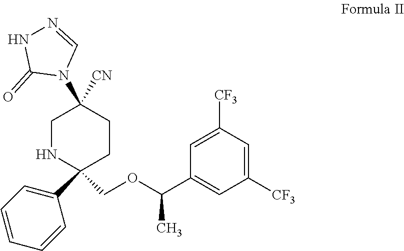 [SMILES: C[C@@H](OC[C@@]1(c2ccccc2)CC[C@](C#N)(n2cn[nH]c2=O)CN1)c1cc(C(F)(F)F)cc(C(F)(F)F)c1]
or a pharmaceutically effective salt thereof and pharmaceutically acceptable excipients.

10. The composition according to claim 9 in the form of a tablet or capsule.

11. The composition according to claim 10 having between 0.10 mgs and 100 mgs of a compound of formula II.

12. A solid dosage form comprising a crystalline compound of formula II in a strength of between about 10 to about 50 mgs.

13. The dosage form according to claim 12 in strengths of 10, 25 and 50 mgs.

14. A method of treating a patient having chronic pruritus with a solid dosage form according to claim 12 wherein the patient is administered a loading dose of 3× said dosage form followed by a daily dose of said dosage form.

15. A method of treating a patient having chronic pruritus with a solid dosage form according to claim 12 wherein the patient is administered a loading dose of 3× said dosage form following by a weekly dose of said dosage form.

16. A method of treating a patient having chronic pruritus with a solid dosage form according to claim 12 wherein the patient is administered a loading dose of 3× said dosage form following by a biweekly (2× per month) dose of said dosage form.

17. A method of treating a patient having chronic pruritus with a therapeutically effective amount of a compound of formula II wherein the patient having a VAS score of greater than 7 improves from a VAS score of greater than 7 to a VAS score of between 0-6.
